SEQUENCE LISTING (158 sequences)

SEQ ID NO: 1 - protein, 183 aa, synthetic construct
  REGION 1..183: note = Synthetic peptide MICA alpha1-alpha2 domain
AAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRT

SEQ ID NO: 2 - protein, 182 aa, synthetic construct
  REGION 1..182: note = Synthetic peptide MICB alpha1-alpha2 domain
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRT

SEQ ID NO: 3 - protein, 187 aa, synthetic construct
  REGION 1..187: note = Synthetic peptide ULBP1 alpha1-alpha2 domain
AAEPHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPMV

SEQ ID NO: 4 - protein, 189 aa, synthetic construct
  REGION 1..189: note = Synthetic peptide ULBP2 alpha1-alpha2 domain
AAEPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPPMV

SEQ ID NO: 5 - protein, 187 aa, synthetic construct
  REGION 1..187: note = Synthetic peptide ULBP3 alpha1-alpha2 domain
AAEPHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPMV

SEQ ID NO: 6 - protein, 187 aa, synthetic construct
  REGION 1..187: note = Synthetic peptide ULBP4 alpha1-alpha2 domain
AAEPHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPPMV

SEQ ID NO: 7 - protein, 198 aa, synthetic construct
  REGION 1..198: note = Synthetic peptide ULBP5 alpha1-alpha2 domain
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPR

SEQ ID NO: 8 - protein, 189 aa, synthetic construct
  REGION 1..189: note = Synthetic peptide ULBP6 alpha1-alpha2 domain
AAEPHSLCYDITVIPKFRPGPRWCAVQGQVDEKTFLHYDCGNKTVTPVSPLGKKLNVTTAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPPMV

SEQ ID NO: 9 - protein, 161 aa, synthetic construct
  REGION 1..161: note = Synthetic peptide OMCP alpha1-alpha2 domain
AAAEPHKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRRTVPPMV

SEQ ID NO: 10 - DNA, 543 bp, synthetic construct
  misc_feature 1..543: note = Synthetic polynucleotide, encoding MICA alpha1-alpha2 domain
  source 1..543: mol_type = other DNA
gagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaaca

SEQ ID NO: 11 - DNA, 561 bp, synthetic construct
  misc_feature 1..561: note = Synthetic polynucleotide, encoding ULBP1 alpha1-alpha2 domain
  source 1..561: mol_type = other DNA
gctgctgagccccactgtctctgctacgactttattataactcctaagtcaagaccagagcctcagtggtgcgaagtacaaggtttggttgacgaaaggcctttccttcactacgattgtgtgaaccataaggcaaaggctttcgccagcctgggtaagaaggtaaacgttactaagacgtgggaggagcagacggaaaccctccgtgatgtggttgactttcttaagggtcagctcctcgatatccaagtggagaatttaatccctatcgaaccgctcactctgcaggccagaatgtcatgcgaacatgaagcacacggtcatggaagaggtagttggcaatttttatttaacggtcaaaaattcctgctgttcgactcaaacaaccgcaaatggactgcgctgcaccctggagctaagaagatgactgaaaaatgggagaagaacagagacgttaccatgttcttccagaagatttccctgggagattgtaagatgtggttagaggagttcttaatgtactgggaacagatgctggaccccacaaaaccccccatggtg

SEQ ID NO: 12 - DNA, 567 bp, synthetic construct
  misc_feature 1..567: note = Synthetic polynucleotide, encoding ULBP2 alpha1-alpha2 domain
  source 1..567: mol_type = other DNA
gctgctgagccccatagtctgtgttacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgcgcgacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcggctggtgccccccccatggtg

SEQ ID NO: 13 - DNA, 561 bp, synthetic construct
  misc_feature 1..561: note = Synthetic polynucleotide, encoding ULBP3 alpha1-alpha2 domain
  source 1..561: mol_type = other DNA
gctgctgagccccacagcttgtggtacaacttcaccattatccacttgccgagacatggccagcagtggtgcgaagtgcaatcgcaagtcgaccaaaaaaacttcttatcatacgactgcggcagcgataaggtcttatctatgggtcatttggaggaacagctctacgcgaccgacgcctggggtaaacagctcgagatgctccgtgaggttggacagaggctgagactggaactggctgacactgagctggaagatttcacacctagtggtccactcacattgcaagtacgcatgagctgcgagtgtgaggccgatggatacattaggggcagctggcagtttagcttcgacggaaggaaattcctgctcttcgacagtaacaataggaagtggactgttgtgcatgctggtgcgcgcagaatgaaggaaaagtgggagaaagatagcggcctgacgaccttcttcaagatggtgtctatgcgtgactgtaagagctggctcagagatttcctcatgcatcgcaagaagaggttagaacctaccgctccccccatggtg

SEQ ID NO: 14 - DNA, 561 bp, synthetic construct
  misc_feature 1..561: note = Synthetic polynucleotide, encoding ULBP4 alpha1-alpha2 domain
  source 1..561: mol_type = other DNA
gctgctgagccccactctctttgcttcaacttcaccattaaatccctgagcaggcctggtcagccgtggtgtgaggcgcaggtctttcttaacaagaatctcttcctccaatacaactctgataacaacatggtaaagccactgggtctcctgggtaaaaaagtctatgctacgagcacttggggagaactcacccagactcttggcgaggtaggaagagacctgcgcatgctcctctgcgatataaagccccaaattaagaccagtgatccgtccactttacaagtcgaaatgttctgccaaagggaggctgaacgctgcaccggagcctcttggcagttcgcgaccaatggcgaaaagtccctcttgttcgatgccatgaatatgacctggaccgtgatcaatcatgaggcctctaagatcaaggagacgtggaaaaaggaccgcggccttgaaaagtactttaggaagttgtctaaaggagactgcgaccattggttacgcgagttcctcggccattgggaagcgatgcccgagccaacggttagcccccccatggtg

SEQ ID NO: 15 - DNA, 567 bp, synthetic construct
  misc_feature 1..567: note = Synthetic polynucleotide, encoding ULBP6 alpha1-alpha2 domain
  source 1..567: mol_type = other DNA
gctgctgagccccactccttatgctatgatatcaccgtgattccaaagttccgaccaggaccccgatggtgcgccgtacagggacaggtcgacgaaaagacttttttacattacgactgcggtaacaagacagtcacaccggtaagtcctttgggaaaaaagttaaacgtaaccactgcttggaaggcccagaaccccgtccttcgagaagtagtggatattttgactgaacagctgcttgacatccagctggaaaactacacacccaaagagcccctgactcttcaagcgcgtatgtcgtgtgagcaaaaggccgaaggacacagctccggatcctggcagttcagtatcgacggtcagaccttcctcctcttcgattcagaaaagcgcatgtggactactgtgcaccccggcgctcgtaagatgaaggaaaagtgggagaatgataaggacgttgccatgagttttcattacattagtatgggagattgcatcggttggctggaagacttcctgatgggtatggatagtacccttgaacctagtgccggagctccccccatggtg

SEQ ID NO: 16 - DNA, 483 bp, synthetic construct
  misc_feature 1..483: note = Synthetic polynucleotide, encoding OMCP alpha1-alpha2 domain
  source 1..483: mol_type = other DNA
gctgctgctgagccccacaagcttgcgttcaacttcaatctggaaataaacggttcagatacccattcaaccgtggacgtttatttagacgattcgcagataatcacctttgacggcaaggacatccgcccaactatcccgttcatgataggtgacgaaatcttccttcctttttataagaatgtgttctctgagttcttcagtttgttccgccgcgtccctacctcaaccccctacgaagacttgacttatttctatgaatgcgactacaccgacaacaaatctacattcgatcaattctacctgtacaacggtgaagagtacaccgtgaagactcaagaggctactaacaagaacatgtggctgaccacttccgagttcagactgaagaagtggttcgacggcgaggactgtatcatgcaccttagaagtttagtgaggaaaatggaagatagcaagagaagaacagtgccccccatggtg

SEQ ID NO: 17 - protein, 139 aa, synthetic construct
  REGION 1..139: note = Synthetic peptide natural NKG2D ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 18 - protein, 139 aa, synthetic construct
  REGION 1..139: note = Synthetic peptide non-natural NKG2D Y152A ectodomain
FLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTV

SEQ ID NO: 19 - protein, 139 aa, synthetic construct
  REGION 1..139: note = Synthetic peptide non-natural NKG2D Y199A ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGAIENCSTPNTYICMQRTV

SEQ ID NO: 20 - protein, 139 aa, synthetic construct
  REGION 1..139: note = Synthetic peptide non-natural NKG2D Y152A/Y199A ectodomain
FLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGAIENCSTPNTYICMQRTV

SEQ ID NO: 21 - protein, 139 aa, synthetic construct
  REGION 1..139: note = Synthetic peptide non-natural NKG2D Y199F eNKG2D1 ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGFIENCSTPNTYICMQRTV

SEQ ID NO: 22 - protein, 139 aa, synthetic construct
  REGION 1..139: note = Synthetic peptide non-natural NKG2D Y152S eNKG2D2 ectodomain
FLNSLFNQEVQIPLTESYCGPCPKNWICYKNNCYQFFDESKNWYESQASCMSQNASLLKVYSKEDQDLLKLVKSSHWMGLVHIPTNGSWQWEDGSILSPNLLTIIEMQKGDCALYASSFKGYIENCSTPNTYICMQRTV

SEQ ID NO: 23 - protein, 139 aa, synthetic construct
  REGION 1..139: note = Synthetic peptide non-natural NKG2D Y152T eNKG2D3 ectodomain
FLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGYIENCSTPNTYICMQRTV

SEQ ID NO: 24 - protein, 139 aa, synthetic construct
  REGION 1..139: note = Synthetic peptide non-natural NKG2D Y152V eNKG2D4 ectodomain
FLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGYIENCSTPNTYICMQRTV

SEQ ID NO: 25 - protein, 139 aa, synthetic construct
  REGION 1..139: note = Synthetic peptide non-natural NKG2D Y152A/Y199F eNKG2D5 ectodomain
FLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTV

SEQ ID NO: 26 - protein, 139 aa, synthetic construct
  REGION 1..139: note = Synthetic peptide non-natural NKG2D Y152L/Y199F eNKG2D6 ectodomain
FLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGFIENCSTPNTYICMQRTV

SEQ ID NO: 27 - protein, 139 aa, synthetic construct
  REGION 1..139: note = Synthetic peptide non-natural NKG2D Y152S/Y199F eNKG2D7 ectodomain
FLNSLFNQEVQIPLTESYCGPCPKNWICYKNNCYQFFDESKNWYESQASCMSQNASLLKVYSKEDQDLLKLVKSSHWMGLVHIPTNGSWQWEDGSILSPNLLTIIEMQKGDCALYASSFKGFIENCSTPNTYICMQRTV

SEQ ID NO: 28 - protein, 139 aa, synthetic construct
  REGION 1..139: note = Synthetic peptide non-natural NKG2D Y152T/Y199F eNKG2D8 ectodomain
FLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGFIENCSTPNTYICMQRTV

SEQ ID NO: 29 - protein, 139 aa, synthetic construct
  REGION 1..139: note = Synthetic peptide non-natural NKG2D Y152V/Y199F eNKG2D9 ectodomain
FLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGFIENCSTPNTYICMQRTV

SEQ ID NO: 30 - protein, 139 aa, synthetic construct
  REGION 1..139: note = Synthetic peptide non-natural NKG2D Y199D eNKG2D10 ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGDIENCSTPNTYICMQRTV

SEQ ID NO: 31 - protein, 139 aa, synthetic construct
  REGION 1..139: note = Synthetic peptide non-natural NKG2D Y199E eNKG2D11 ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGEIENCSTPNTYICMQRTV

SEQ ID NO: 32 - protein, 139 aa, synthetic construct
  REGION 1..139: note = Synthetic peptide non-natural NKG2D Y152D/Y199D eNKG2D12 ectodomain
FLNSLFNQEVQIPLTESYCGPCPKNWICYKNNCYQFFDESKNWYESQASCMSQNASLLKVYSKEDQDLLKLVKSDHWMGLVHIPTNGSWQWEDGSILSPNLLTIIEMQKGDCALYASSFKGDIENCSTPNTYICMQRTV

SEQ ID NO: 33 - protein, 139 aa, synthetic construct
  REGION 1..139: note = Synthetic peptide non-natural NKG2D Y152E/Y199E eNKG2D13 ectodomain
FLNSLFNQEVQIPLTESYCGPCPKNWICYKNNCYQFFDESKNWYESQASCMSQNASLLKVYSKEDQDLLKLVKSEHWMGLVHIPTNGSWQWEDGSILSPNLLTIIEMQKGDCALYASSFKGEIENCSTPNTYICMQRTV

SEQ ID NO: 34 - protein, 139 aa, synthetic construct
  REGION 1..139: note = Synthetic peptide non-natural NKG2D Y152L eNKG2D14 ectodomain
FLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGYIENCSTPNTYICMQRTV

SEQ ID NO: 35 - protein, 139 aa, synthetic construct
  REGION 1..139: note = Synthetic peptide non-natural NKG2D Y152F/Y199F eNKG2D15 ectodomain
FLNSLFNQEVQIPLTESYCGPCPKNWICYKNNCYQFFDESKNWYESQASCMSQNASLLKVYSKEDQDLLKLVKSFHWMGLVHIPTNGSWQWEDGSILSPNLLTIIEMQKGDCALYASSFKGFIENCSTPNTYICMQRTV

SEQ ID NO: 36 - protein, 23 aa, synthetic construct
  REGION 1..23: note = Synthetic peptide MHCI signal sequence
MGLGPVFLLLAGIFPFAPPGAAA

SEQ ID NO: 37 - DNA, 69 bp, synthetic construct
  misc_feature 1..69: note = Synthetic polynucleotide, encoding MHCI signal sequence
  source 1..69: mol_type = other DNA
atgggccttggcccagtgtttctgctgttggcaggcattttcccttttgctccgcccggcgccgcagcc

SEQ ID NO: 38 - protein, 237 aa, synthetic construct
  REGION 1..237: note = Synthetic peptide human IgG1 Fc with IEGR linker
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGR

SEQ ID NO: 39 - DNA, 711 bp, synthetic construct
  misc_feature 1..711: note = Synthetic polynucleotide, encoding human IgG1 Fc with IEGR linker
  source 1..711: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgc

SEQ ID NO: 40 - protein, 376 aa, synthetic construct
  REGION 1..376: note = Synthetic peptide human IgG1 Fc-NKG2D fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 41 - protein, 376 aa, synthetic construct
  REGION 1..376: note = Synthetic peptide human IgG1 Fc-NKG2D Y152A fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTV

SEQ ID NO: 42 - protein, 376 aa, synthetic construct
  REGION 1..376: note = Synthetic peptide human IgG1 Fc-NKG2D Y199A ectodomain
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGAIENCSTPNTYICMQRTV

SEQ ID NO: 43 - protein, 376 aa, synthetic construct
  REGION 1..376: note = Synthetic peptide human IgG1 Fc-NKG2D Y152A/Y199A ectodomain
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGAIENCSTPNTYICMQRTV

SEQ ID NO: 44 - protein, 376 aa, synthetic construct
  REGION 1..376: note = Synthetic peptide human IgG1 Fc-NKG2D Y199F eNKG2D1 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGFIENCSTPNTYICMQRTV

SEQ ID NO: 45 - protein, 376 aa, synthetic construct; identical to SEQ ID NO: 44
  REGION 1..376: note = Synthetic peptide human IgG1 Fc-NKG2D Y152S eNKG2D2 fusion

SEQ ID NO: 46 - protein, 376 aa, synthetic construct
  REGION 1..376: note = Synthetic peptide human IgG1 Fc-NKG2D Y152T eNKG2D3 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGYIENCSTPNTYICMQRTV

SEQ ID NO: 47 - protein, 376 aa, synthetic construct
  REGION 1..376: note = Synthetic peptide human IgG1 Fc-NKG2D Y152V eNKG2D4 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGYIENCSTPNTYICMQRTV

SEQ ID NO: 48 - protein, 376 aa, synthetic construct
  REGION 1..376: note = Synthetic peptide human IgG1 Fc-NKG2D Y152A/Y199F eNKG2D5 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTV

SEQ ID NO: 49 - protein, 376 aa, synthetic construct
  REGION 1..376: note = Synthetic peptide human IgG1 Fc-NKG2D Y152L/Y199F eNKG2D6 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGFIENCSTPNTYICMQRTV

SEQ ID NO: 50 - protein, 376 aa, synthetic construct
  REGION 1..376: note = Synthetic peptide human IgG1 Fc-NKG2D Y152S/Y199F eNKG2D7 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSSHWMGLVHIPTNGSWQWEDGSILSPNLLTIIEMQKGDCALYASSFKGFIENCSTPNTYICMQRTV

SEQ ID NO: 51 - protein, 376 aa, synthetic construct
  REGION 1..376: note = Synthetic peptide human IgG1 Fc-NKG2D Y152T/Y199F eNKG2D8 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGFIENCSTPNTYICMQRTV

SEQ ID NO: 52 - protein, 376 aa, synthetic construct
  REGION 1..376: note = Synthetic peptide human IgG1 Fc-NKG2D Y152V/Y199F eNKG2D9 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGFIENCSTPNTYICMQRTV

SEQ ID NO: 53 - protein, 376 aa, synthetic construct
  REGION 1..376: note = Synthetic peptide human IgG1 Fc-NKG2D Y199D eNKG2D10 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGDIENCSTPNTYICMQRTV

SEQ ID NO: 54 - protein, 376 aa, synthetic construct
  REGION 1..376: note = Synthetic peptide human IgG1 Fc-NKG2D Y199E eNKG2D11 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGEIENCSTPNTYICMQRTV

SEQ ID NO: 55 - protein, 376 aa, synthetic construct
  REGION 1..376: note = Synthetic peptide human IgG1 Fc-NKG2D Y152D/Y199D eNKG2D12 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSDHWMGLVHIPTNGSWQWEDGSILSPNLLTIIEMQKGDCALYASSFKGDIENCSTPNTYICMQRTV

SEQ ID NO: 56 - protein, 376 aa, synthetic construct
  REGION 1..376: note = Synthetic peptide human IgG1 Fc-NKG2D Y152E/Y199E eNKG2D13 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSEHWMGLVHIPTNGSWQWEDGSILSPNLLTIIEMQKGDCALYASSFKGEIENCSTPNTYICMQRTV

SEQ ID NO: 57 - protein, 376 aa, synthetic construct
  REGION 1..376: note = Synthetic peptide human IgG1 Fc-NKG2D Y152L eNKG2D14 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGYIENCSTPNTYICMQRTV

SEQ ID NO: 58 - protein, 376 aa, synthetic construct
  REGION 1..376: note = Synthetic peptide human IgG1 Fc-NKG2D Y152F/Y199F eNKG2D15 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSFHWMGLVHIPTNGSWQWEDGSILSPNLLTIIEMQKGDCALYASSFKGFIENCSTPNTYICMQRTV

SEQ ID NO: 59 - DNA, 1128 bp, synthetic construct
  misc_feature 1..1128: note = Synthetic polynucleotide, encoding human IgG1 Fc-NKG2D fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 60 - DNA, 1128 bp, synthetic construct
  misc_feature 1..1128: note = Synthetic polynucleotide, encoding human IgG1 Fc-NKG2D Y152A fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagctcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 61 - DNA, 1128 bp, synthetic construct
  misc_feature 1..1128: note = Synthetic polynucleotide, encoding human IgG1 Fc-NKG2D Y199A ectodomain
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcataccattggatgggactagtacacattccaacaaatggatcttggcagtgggaagatggctccattctctcacccaacctactaacaataattgaaatgcagaagggagactgtgcactctatgcctcgagctttaaaggcgctatagaaaactgttcaactccaaatacatacatctgcatgcaaaggactgtg

SEQ ID NO: 62 - DNA, 1128 bp, synthetic construct
  misc_feature 1..1128: note = Synthetic polynucleotide, encoding human IgG1 Fc-NKG2D Y152A/Y199A ectodomain
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagctcattggatgggactagtacacattccaacaaatggatcttggcagtgggaagatggctccattctctcacccaacctactaacaataattgaaatgcagaagggagactgtgcactctatgcctcgagctttaaaggcgctatagaaaactgttcaactccaaatacatacatctgcatgcaaaggactgtg

SEQ ID NO: 63 - DNA, 1128 bp, synthetic construct
  misc_feature 1..1128: note = Synthetic polynucleotide, encoding human IgG1 Fc-NKG2D Y199F eNKG2D1 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcataccattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 64 - DNA, 1128 bp, synthetic construct; identical to SEQ ID NO: 63
  misc_feature 1..1128: note = Synthetic polynucleotide, encoding human IgG1 Fc-NKG2D Y152S eNKG2D2 fusion
  source 1..1128: mol_type = other DNA

SEQ ID NO: 65 - DNA, 1128 bp, synthetic construct
  misc_feature 1..1128: note = Synthetic polynucleotide, encoding human IgG1 Fc-NKG2D Y152T eNKG2D3 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcaactcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 66 - DNA, 1128 bp, synthetic construct
  misc_feature 1..1128: note = Synthetic polynucleotide, encoding human IgG1 Fc-NKG2D Y152V eNKG2D4 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagtgcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 67 - DNA, 1128 bp, synthetic construct
  misc_feature 1..1128: note = Synthetic polynucleotide, encoding human IgG1 Fc-NKG2D Y152A/Y199F eNKG2D5 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagctcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 68 - DNA, 1128 bp, synthetic construct
  misc_feature 1..1128: note = Synthetic polynucleotide, encoding human IgG1 Fc-NKG2D Y152L/Y199F eNKG2D6 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcactgcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 69 - DNA, 1128 bp, synthetic construct
  misc_feature 1..1128: note = Synthetic polynucleotide, encoding human IgG1 Fc-NKG2D Y152S/Y199F eNKG2D7 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcaagtcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 70 - DNA, 1128 bp, synthetic construct
  misc_feature 1..1128: note = Synthetic polynucleotide, encoding human IgG1 Fc-NKG2D Y152T/Y199F eNKG2D8 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcaactcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 71 - DNA, 1128 bp, synthetic construct
  misc_feature 1..1128: note = Synthetic polynucleotide, encoding human IgG1 Fc-NKG2D Y152V/Y199F eNKG2D9 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagtgcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 72 - DNA, 1128 bp, synthetic construct
  misc_feature 1..1128: note = Synthetic polynucleotide, encoding human IgG1 Fc-NKG2D Y199D eNKG2D10 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggcgatatagaaaactgttcaactccaaatacatacatctgcatgcaaaggactgtg

SEQ ID NO: 73 - DNA, 1128 bp, synthetic construct
  misc_feature 1..1128: note = Synthetic polynucleotide, encoding human IgG1 Fc-NKG2D Y199E eNKG2D11 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcataccattggatgggactagtacacattccaacaaatggatcttggcagtgggaagatggctccattctctcacccaacctactaacaataattgaaatgcagaagggagactgtgcactctatgcctcgagctttaaaggcgagatagaaaactgttcaactccaaatacatacatctgcatgcaaaggactgtg

SEQ ID NO: 74 - DNA, 1128 bp, synthetic construct
  misc_feature 1..1128: note = Synthetic polynucleotide, encoding human IgG1 Fc-NKG2D Y152D/Y199D eNKG2D12 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagatcattggatgggactagtacacattccaacaaatggatcttggcagtgggaagatggctccattctctcacccaacctactaacaataattgaaatgcagaagggagactgtgcactctatgcctcgagctttaaaggcgatatagaaaactgttcaactccaaatacatacatctgcatgcaaaggactgtg

SEQ ID NO: 75 - DNA, 1128 bp, synthetic construct
  misc_feature 1..1128: note = Synthetic polynucleotide, encoding human IgG1 Fc-NKG2D Y152E/Y199E eNKG2D13 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagagcattggatgggactagtacacattccaacaaatggatcttggcagtgggaagatggctccattctctcacccaacctactaacaataattgaaatgcagaagggagactgtgcactctatgcctcgagctttaaaggcgagatagaaaactgttcaactccaaatacatacatctgcatgcaaaggactgtg

SEQ ID NO: 76 - DNA, 1128 bp, synthetic construct
  misc_feature 1..1128: note = Synthetic polynucleotide, encoding human IgG1 Fc-NKG2D Y152L eNKG2D14 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcactgcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 77 - DNA, 1128 bp, synthetic construct
  misc_feature 1..1128: note = Synthetic polynucleotide, encoding human IgG1 Fc-NKG2D Y152F/Y199F eNKG2D15 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcattccattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 78 - protein, 181 aa, synthetic construct
  REGION 1..181: note = Synthetic peptide MICwed alpha1-alpha2
EPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETKEWTMPQSSRAQTLAMNVRNFLKEDAMETDTHYHAMHADCLQELRRYLKSGVVLRRT

SEQ ID NO: 79 - DNA, 543 bp, synthetic construct
  misc_feature 1..543: note = Synthetic polynucleotide, encoding MICwed alpha1-alpha2
  source 1..543: mol_type = other DNA
gagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccaaggaatggactatgcctcagagctctcgggcacagactctcgctatgaacgttagaaacttccttaaggaggatgctatggagaccgatactcactaccacgccatgcacgccgactgcctccaggaactgcggagatatctgaagtccggcgtggttttgagaagaacc

SEQ ID NO: 80 - protein, 181 aa, synthetic construct
  REGION 1..181: note = Synthetic peptide MIC25 alpha1-alpha2
EPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETLEWTMPQSSRAQTLAMNVRNFLKEDAMETDTHYHAMRADCLSELRRYLKSGVVLRRT

SEQ ID NO: 81 - DNA, 543 bp, synthetic construct
  misc_feature 1..543: note = Synthetic polynucleotide, encoding MIC25 alpha1-alpha2
  source 1..543: mol_type = other DNA
gagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccctcgaatggactatgcctcagagctctcgggcacagactctcgctatgaacgttagaaacttccttaaggaggatgctatggagaccgatactcactaccacgccatgcgcgccgactgcctctctgaactgcggagatatctgaagtccggcgtggttttgagaagaacc

SEQ ID NO: 82 - protein, 330 aa, synthetic construct
  REGION 1..330: note = Synthetic peptide human IgG1 heavy chain CH1-CH2-CH3 D265A/N297A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 990 bp, synthetic construct
  misc_feature 1..990: note = human IgG1 heavy chain CH1-CH2-CH3 D265A/N297A
  source 1..990: mol_type = other DNA
gcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag

SEQ ID NO: 84 - protein, 107 aa, synthetic construct [sequence omitted]

SEQ ID NO: 85 - DNA, 321 bp, synthetic construct
  misc_feature 1..321: note = Synthetic polynucleotide, encoding human kappa light chain
  source 1..321: mol_type = other DNA
agaaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgc

SEQ ID NO: 86 - protein, 120 aa, synthetic construct [sequence omitted]

SEQ ID NO: 87 - DNA, 360 bp, synthetic construct
  misc_feature 1..360: note = Synthetic polynucleotide, encoding trastuzumab VH
  source 1..360: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcg

SEQ ID NO: 88 - protein, 107 aa, synthetic construct [sequence omitted]

SEQ ID NO: 89 - DNA, 321 bp, synthetic construct
  misc_feature 1..321: note = Synthetic polynucleotide, encoding trastuzumab VL
  source 1..321: mol_type = other DNA
gatatccaaatgactcaatcaccatcttcactctccgcgagcgtgggtgatcgggtcaccatcacatgtagggcgagccaagatgtgaataccgccgtcgcgtggtatcaacaaaagccgggaaaagcaccaaaactgcttatatactctgcatccttcctgtactctggggtgccaagccggttctccggtagtagatctggtactgactttacactcactatcagcagtctgcaacctgaggactttgcgacatactattgccagcagcactacacaaccccacctacatttggtcaggggacaaaggtggagatcaag

SEQ ID NO: 90 - protein, 121 aa, synthetic construct [sequence omitted]

SEQ ID NO: 91 - DNA, 363 bp, synthetic construct
  misc_feature 1..363: note = Synthetic polynucleotide, encoding rituximab VH
  source 1..363: mol_type = other DNA
caagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttacccaggtaacggtgacacatcatataaccaaaagtttaagggaaaggcaaccctcacagcggacaagagtagctcaaccgcatacatgcaactgtcaagccttacctccgaagacagcgcagtgtactactgcgccagaagcacctactatgggggtgattggtacttcaacgtctggggggctggcaccacagtgactgtaagcgca

SEQ ID NO: 92 - protein, 106 aa, synthetic construct [sequence omitted]

SEQ ID NO: 93 - DNA, 318 bp, synthetic construct
  misc_feature 1..318: note = Synthetic polynucleotide, encoding rituximab VL
  source 1..318: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaag

SEQ ID NO: 94 - protein, 450 aa, synthetic construct
  REGION 1..450: note = Synthetic peptide trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1350 bp, synthetic construct
  misc_feature 1..1350: note = Synthetic polynucleotide, encoding trastuzumab heavy chain
  source 1..1350: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcggcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag

SEQ ID NO: 96 - protein, 213 aa, synthetic construct
  REGION 1..213: note = Synthetic peptide trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - DNA, 642 bp, synthetic construct
  misc_feature 1..642: note = Synthetic polynucleotide, encoding trastuzumab light chain
  source 1..642: mol_type = other DNA
gatatccaaatgactcaatcaccatcttcactctccgcgagcgtgggtgatcgggtcaccatcacatgtagggcgagccaagatgtgaataccgccgtcgcgtggtatcaacaaaagccgggaaaagcaccaaaactgcttatatactctgcatccttcctgtactctggggtgccaagccggttctccggtagtagatctggtactgactttacactcactatcagcagtctgcaacctgaggactttgcgacatactattgccagcagcactacacaaccccacctacatttggtcaggggacaaaggtggagatcaagaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgctag

SEQ ID NO: 98 - protein, 451 aa, synthetic construct
  REGION 1..451: note = Synthetic peptide rituximab heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 1353 bp, synthetic construct
  misc_feature 1..1353: note = Synthetic polynucleotide, encoding rituximab heavy chain
  source 1..1353: mol_type = other DNA
caagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttacccaggtaacggtgacacatcatataaccaaaagtttaagggaaaggcaaccctcacagcggacaagagtagctcaaccgcatacatgcaactgtcaagccttacctccgaagacagcgcagtgtactactgcgccagaagcacctactatgggggtgattggtacttcaacgtctggggggctggcaccacagtgactgtaagcgcagcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag

SEQ ID NO: 100 - protein, 212 aa, synthetic construct
  REGION 1..212: note = Synthetic peptide rituximab light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - DNA, 639 bp, synthetic construct
  misc_feature 1..639: note = Synthetic polynucleotide, encoding rituximab light chain
  source 1..639: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgctag

SEQ ID NO: 102 - protein, 634 aa, synthetic construct
  REGION 1..634: note = Synthetic peptide trastuzumab HC_MICwed
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSEPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETKEWTMPQSSRAQTLAMNVRNFLKEDAMETDTHYHAMHADCLQELRRYLKSGVVLRRT

SEQ ID NO: 103 - DNA, 1902 bp, synthetic construct
  misc_feature 1..1902: note = Synthetic polynucleotide, encoding trastuzumab HC_MICwed
  source 1..1902: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcggcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccaaggaatggactatgcctcagagctctcgggcacagactctcgctatgaacgttagaaacttccttaaggaggatgctatggagaccgatactcactaccacgccatgcacgccgactgcctccaggaactgcggagatatctgaagtccggcgtggttttgagaagaacc

SEQ ID NO: 104 - protein, 634 aa, synthetic construct
  REGION 1..634: note = Synthetic peptide trastuzumab HC_MIC25
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSEPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETLEWTMPQSSRAQTLAMNVRNFLKEDAMETDTHYHAMRADCLSELRRYLKSGVVLRRT

SEQ ID NO: 105 - DNA, 1902 bp, synthetic construct
  misc_feature 1..1902: note = Synthetic polynucleotide, encoding trastuzumab HC_MIC25
  source 1..1902: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcggcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccctcgaatggactatgcctcagagctctcgggcacagactctcgctatgaacgttagaaacttccttaaggaggatgctatggagaccgatactcactaccacgccatgcgcgccgactgcctctctgaactgcggagatatctgaagtccggcgtggttttgagaagaacc

SEQ ID NO: 106 - protein, 404 aa, synthetic construct
  REGION 1..404: note = Synthetic peptide rituximab LC_ULBP2.wt
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPS

SEQ ID NO: 107 - DNA, 1212 bp, synthetic construct
  misc_feature 1..1212: note = Synthetic polynucleotide, encoding rituximab LC_ULBP2.wt
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgcgcgacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcg

SEQ ID NO: 108 - protein, 187 aa, synthetic construct
  REGION 1..187: note = Synthetic peptide ULPB2 alpha1-alpha2 variant R80W
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPPMV

SEQ ID NO: 109 - DNA, 540 bp, synthetic construct
  misc_feature 1..540: note = Synthetic polynucleotide, encoding ULPB2 alpha1-alpha2 variant R80W
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcg

SEQ ID NO: 110 - DNA, 513 bp, synthetic construct
  misc_feature 1..513: note = Synthetic polynucleotide, encoding ULPB2 alpha1-alpha2 variant R80W NNK library
  misc_feature 457..458: note = n is a, c, g, or t
  misc_feature 460..461: note = n is a, c, g, or t
  misc_feature 463..464: note = n is a, c, g, or t
  misc_feature 466..467: note = n is a, c, g, or t
  misc_feature 469..470: note = n is a, c, g, or t
  source 1..513: mol_type = other DNA
gagccccatagcctcagctatgacattacggtgattcccaaatttcgcccaggaccacgttggtgcgccgtccagggtcaggtagatgaaaagactttcctgcattatgattgcggcaataaaaccgtgacgccggtatcgccgttaggcaaaaaattgaatgtcacgacagcgtggaaagcacagaacccggtgttgcgcgaggtagtcgatattttgacggaacaactctgggacattcagctcgagaattacaccccaaaagaaccgctgacgctgcaagcgcgtatgtcgtgcgaacaaaaagcagaaggtcactctagcgggagttggcagttttccttcgatgggcagatttttctgctgtttgattcggagaaacgcatgtggactacagtccacccgggtgcccggaaaatgaaagagaagtgggagaatgataaagtggtggccactnnknnknnknnknnktccatgggcgattgcattggctggttagaggattttctcatg

SEQ ID NO: 111 - protein, 180 aa, synthetic construct
  REGION 1..180: note = Synthetic peptide, ULBP2 alpha1-alpha2 variant ULBP2.C
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATILWQTSMGDCIGWLEDFLMGMDSTLEPS

SEQ ID NO: 112 - DNA, 540 bp, synthetic construct
  misc_feature 1..540: note = Synthetic polynucleotide, encoding ULBP2 alpha1-alpha2 variant ULBP2.C
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactattctgtggcagacttcgatgggagactgcatcggttggctggaagatttcctcatgggtatggactccactttggagccatcg

SEQ ID NO: 113 - protein, 180 aa, synthetic construct
  REGION 1..180: note = Synthetic peptide, ULBP2 alpha1-alpha2 variant ULBP2.R
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLLWGWSMGDCIGWLEDFLMGMDSTLEPS

SEQ ID NO: 114 - DNA, 540 bp, synthetic construct
  misc_feature 1..540: note = Synthetic polynucleotide, encoding ULBP2 alpha1-alpha2 variant ULBP2.R
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactttgttgtgggggtggtcgatgggagactgcatcggttggctggaagatttcctcatgggtatggactccactttggagccatcg

SEQ ID NO: 115 - protein, 180 aa, synthetic construct
  REGION 1..180: note = Synthetic peptide, ULBP2 alpha1-alpha2 variant ULBP2.AA
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATMFWSWSMGDCIGWLEDFLMGMDSTLEPS

SEQ ID NO: 116 - DNA, 540 bp, synthetic construct
  misc_feature 1..540: note = Synthetic polynucleotide, encoding ULBP2 alpha1-alpha2 variant ULBP2.AA
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactatgttttggagttggtcgatgggagactgcatcggttggctggaagatttcctcatgggtatggactccactttggagccatcg

SEQ ID NO: 117 - protein, 180 aa, synthetic construct
  REGION 1..180: note = Synthetic peptide, ULBP2 alpha1-alpha2 variant ULBP2.AB
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMWQWSMGDCIGWLEDFLMGMDSTLEPS

SEQ ID NO: 118 - DNA, 540 bp, synthetic construct
  misc_feature 1..540: note = Synthetic polynucleotide, encoding ULBP2 alpha1-alpha2 variant ULBP2.AB
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactcttatgtggcagtggtcgatgggagactgcatcggttggctggaagatttcctcatgggtatggactccactttggagccatcg

SEQ ID NO: 119 - protein, 404 aa, synthetic construct
  REGION 1..404: note = Synthetic peptide rituximab LC_ULBP2.C
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATILWQTSMGDCIGWLEDFLMGMDSTLEPS

SEQ ID NO: 120 - DNA, 1212 bp, synthetic construct
  misc_feature 1..1212: note = Synthetic polynucleotide, encoding rituximab LC_ULBP2.C
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactattctgtggcagacttcgatgggagactgcatcggttggctggaagatttcctcatgggtatggactccactttggagccatcg

SEQ ID NO: 121 - protein, 404 aa, synthetic construct
  REGION 1..404: note = Synthetic peptide rituximab LC_ULBP2.R
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLLWGWSMGDCIGWLEDFLMGMDSTLEPS

SEQ ID NO: 122 - DNA, 1212 bp, synthetic construct
  misc_feature 1..1212: note = Synthetic polynucleotide, encoding rituximab LC_ULBP2.R
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactttgttgtgggggtggtcgatgggagactgcatcggttggctggaagatttcctcatgggtatggactccactttggagccatcg

SEQ ID NO: 123 - protein, 404 aa, synthetic construct
  REGION 1..404: note = Synthetic peptide rituximab LC_ULBP2.AA
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATMFWSWSMGDCIGWLEDFLMGMDSTLEPS

SEQ ID NO: 124 - DNA, 1212 bp, synthetic construct
  misc_feature 1..1212: note = Synthetic polynucleotide, encoding rituximab LC_ULBP2.AA
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactatgttttggagttggtcgatgggagactgcatcggttggctggaagatttcctcatgggtatggactccactttggagccatcg

SEQ ID NO: 125 - protein, 404 aa, synthetic construct
  REGION 1..404: note = Synthetic peptide rituximab LC_ULBP2.AB
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMWQWSMGDCIGWLEDFLMGMDSTLEPS

SEQ ID NO: 126 - DNA, 1212 bp, synthetic construct
  misc_feature 1..1212: note = Synthetic polynucleotide, encoding rituximab LC_ULBP2.AB
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactcttatgtggcagtggtcgatgggagactgcatcggttggctggaagatttcctcatgggtatggactccactttggagccatcg

SEQ ID NO: 127 - protein, 180 aa, synthetic construct
  REGION 1..180: note = Synthetic peptide, ULBP2 alpha1-alpha2 variant ULBP2.S3
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 128 - DNA, 540 bp, synthetic construct
  misc_feature 1..540: note = Synthetic polynucleotide, encoding ULBP2 alpha1-alpha2 variant ULBP2.S3
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactaagctttatctttggtcgatgggagactgcatcggttggctggaagatttcctcatgggtatggactccactttggagccatcg

SEQ ID NO: 129 - protein, 404 aa, synthetic construct
  REGION 1..404: note = Synthetic peptide rituximab LC_ULBP2.S3
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 130 - DNA, 1212 bp, synthetic construct
  misc_feature 1..1212: note = Synthetic polynucleotide, encoding rituximab LC_ULBP2.S3
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactaagctttatctttggtcgatgggagactgcatcggttggctggaagatttcctcatgggtatggactccactttggagccatcg

SEQ ID NO: 131 - protein, 634 aa, synthetic construct
  REGION 1..634: note = Synthetic peptide rituximab HC_ULBP2.R80W
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPS

SEQ ID NO: 132 - DNA, 1902 bp, synthetic construct
  misc_feature 1..1902: note = Synthetic polynucleotide, encoding rituximab HC_ULBP2.R80W
  source 1..1902: mol_type = other DNA
caagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttacccaggtaacggtgacacatcatataaccaaaagtttaagggaaaggcaaccctcacagcggacaagagtagctcaaccgcatacatgcaactgtcaagccttacctccgaagacagcgcagtgtactactgcgccagaagcacctactatgggggtgattggtacttcaacgtctggggggctggcaccacagtgactgtaagcgcagcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcg

SEQ ID NO: 133 - protein, 405 aa, synthetic construct
  REGION 1..405: note = Synthetic peptide trastuzumab LC_ULBP2.R
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLLWGWSMGDCIGWLEDFLMGMDSTLEPS

SEQ ID NO: 134 - DNA, 1215 bp, synthetic construct
  misc_feature 1..1215: note = Synthetic polynucleotide, encoding trastuzumab LC_ULBP2.R
  source 1..1215: mol_type = other DNA
gatatccaaatgactcaatcaccatcttcactctccgcgagcgtgggtgatcgggtcaccatcacatgtagggcgagccaagatgtgaataccgccgtcgcgtggtatcaacaaaagccgggaaaagcaccaaaactgcttatatactctgcatccttcctgtactctggggtgccaagccggttctccggtagtagatctggtactgactttacactcactatcagcagtctgcaacctgaggactttgcgacatactattgccagcagcactacacaaccccacctacatttggtcaggggacaaaggtggagatcaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactttgttgtgggggtggtcgatgggagactgcatcggttggctggaagatttcctcatgggtatggactccactttggagccatcg

SEQ ID NO: 135 - protein, 156 aa, synthetic construct
  REGION 1..156: note = Synthetic peptide NKG2D.wt ectodomain
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 136 - DNA, 468 bp, synthetic construct
  misc_feature 1..468: note = Synthetic polynucleotide, encoding NKG2D.wt ectodomain
  source 1..468: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagctaccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtg

SEQ ID NO: 137 - protein, 156 aa, synthetic construct
  REGION 1..156: note = Synthetic peptide NKG2D.YA ectodomain
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTV

SEQ ID NO: 138 - DNA, 468 bp, synthetic construct
  misc_feature 1..468: note = Synthetic polynucleotide, encoding NKG2D.YA ectodomain
  source 1..468: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtg

SEQ ID NO: 139 - protein, 156 aa, synthetic construct
  REGION 1..156: note = Synthetic peptide NKG2D.AF ectodomain
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTV

SEQ ID NO: 140 - DNA, 468 bp, synthetic construct
  misc_feature 1..468: note = Synthetic polynucleotide, encoding NKG2D.AF ectodomain
  source 1..468: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggcttcatcgagaactgcagcacccctaacacctacatctgtatgcagcggaccgtg

SEQ ID NO: 141 - protein, 69 aa, synthetic construct
  REGION 1..69: note = Synthetic peptide CD8alpha hinge and transmembrane domain
TTTPAPRPPTPAPTIASQPLSLRPEACRPAAGGAVHTRGLDFACDIYIWAPLAGTCGVLLLSLVITLYC

SEQ ID NO: 142 - DNA, 207 bp, synthetic construct
  misc_feature 1..207: note = Synthetic polynucleotide, encoding CD8alpha hinge and transmembrane domain
  source 1..207: mol_type = other DNA
accaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgc

SEQ ID NO: 143 - protein, 42 aa, synthetic construct
  REGION 1..42: note = Synthetic peptide 4-1BB
KRGRKKLLYIFKQPFMRPVQTTQEEDGCSCRFPEEEEGGCEL

SEQ ID NO: 144 - DNA, 126 bp, synthetic construct
  misc_feature 1..126: note = Synthetic polynucleotide, encoding 4-1BB
  source 1..126: mol_type = other DNA
aagcggggcagaaagaagctgctgtacatctttaagcagcccttcatgcggcccgtgcagaccacacaagaggaagatggctgctcctgcagattccccgaggaagaagaaggcggctgcgagctg

SEQ ID NO: 145 - protein, 112 aa, synthetic construct
  REGION 1..112: note = Synthetic peptide CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - DNA, 336 bp, synthetic construct
  misc_feature 1..336: note = Synthetic polynucleotide, encoding CD3zeta
  source 1..336: mol_type = other DNA
agagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 147 - protein, 239 aa, synthetic construct
  REGION 1..239: note = Synthetic peptide EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 148 - DNA, 717 bp, synthetic construct
  misc_feature 1..717: note = Synthetic polynucleotide, encoding EGFP
  source 1..717: mol_type = other DNA
atggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 149 - protein, 473 aa, synthetic construct
  REGION 1..473: note = Synthetic peptide CD8hingeTM_4-1BB_CD3zeta_EGFP
TTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 150 - DNA, 1419 bp, synthetic construct
  misc_feature 1..1419: note = Synthetic polynucleotide, encoding CD8hingeTM_4-1BB_CD3zeta_EGFP
  source 1..1419: mol_type = other DNA
accaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 151 - protein, 629 aa, synthetic construct
  REGION 1..629: note = Synthetic peptide NKG2D.wt_CD8hingeTM_4-1BB_CD3zeta_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 152 - DNA, 1887 bp, synthetic construct
  misc_feature 1..1887: note = Synthetic polynucleotide, encoding NKG2D.wt_CD8hingeTM_4-1BB_CD3zeta_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagctaccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 153 - protein, 629 aa, synthetic construct
  REGION 1..629: note = Synthetic peptide NKG2D.YA_CD8hingeTM_4-1BB_CD3zeta_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 154 - DNA, 1887 bp, synthetic construct
  misc_feature 1..1887: note = Synthetic polynucleotide, encoding NKG2D.YA_CD8hingeTM_4-1BB_CD3zeta_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 155 - protein, 629 aa, synthetic construct
  REGION 1..629: note = Synthetic peptide NKG2D.AF_CD8hingeTM_4-1BB_CD3zeta_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 156 - DNA, 1887 bp, synthetic construct
  misc_feature 1..1887: note = Synthetic polynucleotide, encoding NKG2D.AF_CD8hingeTM_4-1BB_CD3zeta_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggcttcatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 157 - protein, 11 aa, synthetic construct
  REGION 1..11: note = Synthetic
APTSSSGGGGS

SEQ ID NO: 158 - protein, 4 aa, synthetic construct
  REGION 1..4: note = Synthetic
GGGS

(19) United States
(12) Patent Application Publication
(10) Pub. No.: US 2025/0109178 A1
(43) Pub. Date: Apr. 3, 2025

(54) MODIFIED A1-A2 DOMAINS OF NON-NATURAL NKG2D LIGANDS THAT BIND NON-NATURAL NKG2D RECEPTORS

(71) Applicant: XYPHOS BIOSCIENCES INC., South San Francisco, CA (US)

(72) Inventors: Kaman Kim, South San Francisco, CA (US); Kyle Landgraf, South San Francisco, CA (US)

(21) Appl. No.: 18/919,781

(22) Filed: Oct. 18, 2024

Related U.S. Application Data
(62) Division of application No. 16/366,320, filed on Mar. 27, 2019, now Pat. No. 12,145,976.
(60) Provisional application No. 62/648,636, filed on Mar. 27, 2018.

(51) Int. Cl. C07K 14/705; A61K 35/17; A61K 39/00; A61P 35/00; C07K 14/74; C07K 16/28

(52) U.S. Cl. CPC C07K 14/7056; A61K 35/17; A61K 40/11; A61K 40/31; A61K 40/4224; A61P 35/00; C07K 14/70539; C07K 16/2833; C07K 16/2851

(57) ABSTRACT

This present disclosure relates generally to the production of polypeptides comprised of modified α1-α2 domains of NKG2D ligands which bind specifically to a non-natural ectodomain of a non-natural NKG2D receptor and wherein heterologous molecules are attached to the modified α1-α2 domains of NKG2D ligands. The present disclosure further relates to modified α1-α2 domains of NKG2D ligands attached to heterologous molecules including polypeptides, and in some embodiments, antibodies or fragments of antibodies. The present disclosure also relates to modified forms of the NKG2D receptor engineered to provide a combination of enhanced and diminished binding to non-natural and natural versions of NKG2D ligands, respectively.

### BACKGROUND OF THE INVENTION

### Field of the Invention

This application relates generally to the production of polypeptides comprised of modified α1-α2 domains of NKG2D ligands which bind specifically to a non-natural ectodomain of a non-natural NKG2D receptor and wherein heterologous molecules are attached to the modified α1-α2 domains of NKG2D ligands.

### Background Information

NKG2D is an activating receptor expressed as a type II homodimeric integral protein on the surface of Natural Killer (NK) cells and certain T cells. When bound to one of its eight natural ligands expressed primarily on the surfaces of distressed cells, the NKG2D activates the NK cell to kill the stressed cell, or when on T cells, the ligand-occupied NKG2D co-stimulates the T cell to carry out its effector function. The three-dimensional structures have been solved for the ectodomain of human natural NKG2D, several of its soluble natural ligands and, in some cases, the bound complex of soluble ligand and receptor ectodomain. The monomeric α1-α2 domains of NKG2D ligands bind specifically to the two ectodomains of the natural NKG2D homodimer.

### SUMMARY OF THE INVENTION

The present disclosure relates to modified α1-α2 domains of NKG2D ligands attached to heterologous molecules including polypeptides, in some embodiments antibodies or fragments of antibodies. The invention also relates to modified forms of the NKG2D receptor engineered to provide a combination of enhanced and diminished binding to non-natural and natural versions of NKG2D ligands, respectively.

### BRIEF DESCRIPTION OF THE DRAWINGS

FIG. 1A. Alignment of the natural NKG2D.wt ectodomain (SEQ ID NO: 17) along with the NKG2D.YA (SEQ ID NO: 18) and NKG2D.AF (SEQ ID NO: 25) non-natural variants. Indicated are the locations of Y152 and Y199 and highlighted in gray are the mutated residues present in the non-natural variants.

FIG. 1B. Alignment of the α1-α2 domain of natural/wild-type ULBP2 (SEQ ID NO: 4) and non-natural variants of ULBP2 including ULBP2.R80W (SEQ ID NO: 108). Highlighted in gray are the residues critical for binding of non-natural ULBP2 variants to the non-natural NKG2D.YA or NKG2D.AF receptors. Indicated are the locations of residue R80 as well as the M154-F159 region that was explored for orthoganal variants binding to NKG2D.YA (ULBP2.S3, SEQ ID NO.: 127) or NKG2D.AF (ULBP2.C, SEQ ID NO.: 111; ULBP2.R, SEQ ID NO.: 113; ULPB2.AA, SEQ ID NO.: 115; and ULBP2.AB, SEQ ID NO.: 117).

FIG. 2. Summary of candidate, non-natural Fc-eNKG2D variant mutations and protein aggregation properties determined by Size-Exclusion Chromatography (SEC), as depicted in FIGS. 3 and 4.

FIG. 3. Size-exclusion chromatography comparison of non-natural Fc-NKG2D fusion proteins analyzed on an Akta HiLoad 16/600 Superdex 200 column. Migration of correctly assembled material was exemplified by a discrete, symmetrical peak that eluted at higher volumes while aggregated material eluted sooner at lower volumes. The site and nature of the modifications are indicated by the amino acid numbers Y152, Y199, or both (SEQ ID NOs: 48, 43, 42, 58, 41, and 40 starting from the top of the figure).

FIG. 4. Size-exclusion chromatography profiles of non-natural Fc-eNKG2D variants with one or two amino acid changes were analyzed on an Akta Superdex 200 Increase 10/300 GL column. Migration of correctly assembled material is exemplified by a discrete, symmetrical peak eluting at higher volumes while aggregated material—characterized by a low amplitude broad peak or series of peaks—eluted at lower volumes. The letters in parentheses represent the amino acids at positions 152 and 199 (SEQ ID NOs: 57, 56, 55, 54, 53, 52, 51, 50, 49, 48, 47, 46, 45, 44, and 40 in order from the top), respectively.

FIG. 5. Percent saturation (Rmax) of eNKG2D variants normalized to wild-type NKG2D binding by either MICwed-MicAbody or to MIC25-MicAbody. Wild-type Fc-NKG2D and each Fc-eNKG2D receptor were captured on AHC biosensors then exposed to trastuzumab-specific MicAbodies at 20 nM. Dissociation kinetics were monitored and the Rmax values of the Fc-eNKG2D fusions ranked. Those samples not tested (nt) were due to either severe aggregation or inadequate amount of material expressed or recovered after SEC fractionation.

FIG. 6. ELISA binding of ULBP2 wild-type, MICwed- and MIC25-rituximab MicAbodies to Fc-eNKG2D candidates. Key is indicated at the top of the figure, but since many of the curves overlapped, individual curves were also labeled in each graph.

FIG. 7. EC50 values (nM) for Fc-eNKG2D ELISAs shown in FIG. 4. nt=not tested; nb=no binding or very low binding even at 300 nM so EC50 value not calculated.

FIG. 8. Binding of eNKG2D variants to wild-type ligands. Wild-type ligands (all in Fc-fusion format) were captured onto Octet AHC biosensors, and each natural NKG2D, NKG2D.Y152A, or eNKG2D5 (Y152A/Y199F) as an Fc-fusion was titrated from 300 nM to 0.41 nM. Maximal binding responses were quantified by Octet. (Note the different ordinates for each graph).

FIG. 9. Subset of combinatorial mutations within ULBP2 that resulted in phage clones with selective binding to NKG2D.AF versus natural NKG2D.wt as verified by spot ELISA. Mutants were ranked by frequency of appearance among the selected phages.

FIG. 10. Titration ELISA of individual phage variants to confirm selective binding to Fc-NKG2D.AF and reduced or eliminated binding to Fc-NKG2D.wt. Mutations are detailed in FIG. 11.

FIG. 11. Specificity of NKG2D.AF-selected ULBP2 variants in rituximab-MicAbody format retained their binding to NKG2D.AF by quantitative ELISA. The specific amino acid modifications of each ULBP2 variant are shown as are the ratios of their binding to Fc-NKG2D.wt fusion versus Fc-NKG2D.AF fusion. The amino acid residue locations of ULBP2 are those of FIG. 1B.

FIG. 12. Selected mutations at the indicated amino acid locations of ULBP2.R80W (FIG. 1B; SEQ ID NO: 108) that resulted in Y152A-specific phage clones.

FIG. 13. ELISA data of four non-natural α1-α2 ULBP2 variant MicAbodies binding to NKG2D.wt, NKG2D.YA, and NKG2D.AF. The Fc-NKG2D variants were used as capture agents. MicAbodies were titrated in and detected with HRP-conjugated anti-human kappa.

FIG. 14: ULBP2.C (SEQ ID NO: 111), ULBP2.R (SEQ ID NO: 113), ULBP2.AA (SEQ ID NO: 115), and ULBP2.AB (SEQ ID NO: 117) were examined for changes in peptide-MHCI immunogenicity relative to wild-type ULBP2 (SEQ ID NO: 4) using the NetMHC4.0 Server, querying against the HLA supertype representatives. For the input sequence, the variable region (residues 154-159 according to alignment in FIG. 1B) for each variant along with upstream and downstream nine residues (24 residues total) was entered and 9-mer peptide windows examined for predicted immunogenicity. Dark gray boxes correspond to peptides strongly predicted to bind the MHCI pocket (defined as having % rank<0.5) and therefore have a strong chance of being presented. Light gray boxes correspond to predicted weak binders (% rank<2). See Example 5 text for additional details.

FIG. 15: ULBP2.C (SEQ ID NO: 111), ULBP2.R (SEQ ID NO: 113), ULBP2.AA (SEQ ID NO: 115), and ULBP2.AB (SEQ ID NO: 117) were examined for changes in peptide-MHC class II immunogenicity relative to wild-type ULBP2 (SEQ ID NO: 4) using the NetMHCll 2.3 Server, querying against HLA-DR, HLA-DQ, HLA-DP. For the input sequence, the variable region (residues 154-159 according to alignment in FIG. 1B) for each variant along with upstream and downstream 15 residues (36 residues total) was entered and 15-mer peptide windows examined for predicted immunogenicity. Dark gray boxes correspond to peptides strongly predicted to bind the MHCII pocket and therefore likely to be presented and immunogenic. Light gray boxes correspond to predicted weak binders.

FIGS. 16A and 16B. ELISA-measured binding of rituximab-MicAbodies comprised of ULBP2.wt (wild-type), ULBP2.R80W (which has enhanced affinity for wild-type NKG2D), ULPB2.S3 (NKG2D.YA-selected orthogonal variant), or ULBP2.R (NKG2D.AF-selected orthogonal variant) to natural NKG2D.wt, to NKG2D.YA, and to NKG2D.AF. FIG. 16A: ELISA curves. The reduction in 458 nm absorption for some assays at higher concentrations is an artifact that is often seen with high affinity engagers at higher concentrations due to precipitation of the TMB-Ultra ELISA development reagent. FIG. 16B: EC50 values (reported in nM) as determined in GraphPad Prism based upon the curves in FIG. 16A. nd=not determined due to the lack of relationship between increased concentration and binding.

FIGS. 17A, 17B and 17C. In vitro cytolytic assays with CD8 effector cells that were either untransduced or transduced with NKG2D.wt, NKG2D.YA, or NKG2D.AF CAR constructs consisting of the CD8a hinge/transmembrane domain and intracellular 4-1BB and CD3zeta signaling domains. Target cells were pre-loaded with calcein and exposed to effector cells at increasing effector to target (E:T) ratios. Released calcein was quantified after five hours. FIG. 17A: Cell lysis of HeLa cells. FIG. 17B: lysis of HeLa cells transfected to over-express surface ULBP1. FIG. 17C: cytolysis of HeLa cells expressing non-natural ULBP2.R on their surface. Error bars correspond to standard deviation of technical replicates in the experiment.

FIGS. 18A and 18B. MicAbody directed cytolysis of tumor lines by NKG2D-CAR CD8 T cells. FIG. 18A: Ramos cells—which express CD20, the target of rituximab—were preloaded with calcein and exposed to NKG2D.AF- or NKG2D.YA-CAR cells at an E:T ratio of 20:1 along with increasing concentrations of either ULBP2.S2 or ULBP2.R rituximab-Micabody. The level of cytolysis was quantified after two hours of coincubation. FIG. 18B: The mouse tumor line CT26 transfected to express human Her2 was used as a cytolysis target in parallel with Ramos cells. NKG2D.AF-CAR CD8 T cells were pre-armed with a saturating concentration (5 nM) of rituximab-ULBP2.R, trastuzumab-ULBP2.R, or an equimolar mixture of the two MicAbodies. Unbound MicAbody was removed by washing and CD8 cells added to target cells at two different E:T ratios. Cytolysis was measure after two hours.

### DETAILED DESCRIPTION OF THE INVENTION

Natural killer (NK) cells and certain (CD8+ αβ and γδ) T cells of the immune system have important roles in humans and other mammals as first-line, innate defense against neoplastic and infected cells (Cerwenka, A., and L. L. Lanier. 2001. NK cells, viruses and cancer. Nat. Rev. Immunol. 1:41-49). NK cells and certain T cells exhibit on their surfaces NKG2D, a prominent, homodimeric, surface immunoreceptor responsible for recognizing a target cell and activating the innate defense against the pathologic cell (Lanier, L L, 1998. NK cell receptors. Ann. Rev. Immunol. 16: 359-393; Houchins J P et al. 1991. DNA sequence analysis of NKG2, a family of related cDNA clones encoding type II integral membrane proteins on human NK cells. J. Exp. Med. 173: 1017-1020; Bauer, S et al., 1999. Activation of NK cells and T cells by NKG2D, a receptor for stress-inducible MICA. Science 285: 727-730). The human NKG2D molecule possesses a C-type lectin-like extracellular (ecto-)domain that binds to its eight distinct cognate ligands, the most studied ligands being the 84% sequence identical or homologous, monomeric MICA and MICB, polymorphic analogs of the Major Histocompatibility Complex (MHC) Class I chain-related glycoproteins (MIC) (Weis et al. 1998. The C-type lectin superfamily of the immune system. Immunol. Rev. 163: 19-34; Bahram et al. 1994. A second lineage of mammalian MHC class I genes. PNAS 91:6259-6263; Bahram et al. 1996a. Nucleotide sequence of the human MHC class I MICA gene. Immunogenetics 44: 80-81; Bahram and Spies T A. 1996. Nucleotide sequence of human MHC class I MICB cDNA. Immunogenetics 43: 230-233). Non-pathologic expression of MICA and MICB is restricted to intestinal epithelium, keratinocytes, endothelial cells and monocytes, but aberrant surface expression of these MIC proteins occurs in response to many types of cellular stress such as proliferation, oxidation and heat shock and marks the cell as pathologic (Groh et al. 1996. Cell stress-regulated human MHC class I gene expressed in GI epithelium. PNAS 93: 12445-12450; Groh et al. 1998. Recognition of stress-induced MHC molecules by intestinal γδ T cells. Science 279: 1737-1740; Zwirner et al. 1999. Differential expression of MICA by endothelial cells, fibroblasts, keratinocytes and monocytes. Human Immunol. 60: 323-330). Pathologic expression of MIC proteins also seems involved in some autoimmune diseases (Ravetch, JV and Lanier L L. 2000. Immune Inhibitory Receptors. Science 290: 84-89; Burgess, S J. 2008. Immunol. Res. 40: 18-34). The differential regulation of NKG2D ligands, such as the polymorphic MICA and MICB, is important to provide the immunity system with a means to identify and respond to a broad range of emergency cues while still protecting healthy cells from unwanted attack (Stephens H A, (2001) MICA and MICB genes: can the enigma of their polymorphism be resolved? Trends Immunol. 22: 378-85; Spies, T. 2008. Regulation of NKG2D ligands: a purposeful but delicate affair. Nature Immunol. 9: 1013-1015).

Viral infection is a common inducer of MIC protein expression and identifies the viral-infected cell for NK or T cell attack (Groh et al. 1998; Groh et al. 2001. Co-stimulation of CD8+αβT cells by NKG2D via engagement by MIC induced on virus-infected cells. Nat. Immunol. 2: 255-260; Cerwenka, A., and L. L. Lanier. 2001). In fact, to avoid such an attack on its host cell, cytomegalovirus and other viruses have evolved mechanisms that prevent the expression of MIC proteins on the surface of the cell they infect in order to escape targeting by the innate immunity system (Lodoen, M., K. Ogasawara, J. A. Hamerman, H. Arase, J. P. Houchins, E. S. Mocarski, and L. L. Lanier. 2003. NKG2D-mediated NK cell protection against cytomegalovirus is impaired by gp40 modulation of RAE-1 molecules. J. Exp. Med. 197:1245-1253; Stern-Ginossar et al., (2007) Host immune system gene targeting by viral miRNA. Science 317: 376-381; Stern-Ginossar et al., (2008) Human microRNAs regulate stress-induced immune responses mediated by the receptor NKG2D. Nature Immunology 9: 1065-73; Slavuljica, I A Busche, M Babic, M Mitrovic, I Gašparovic, Ð Cekinovic, E Markova Car, E P Pugel, A Cikovic, V J Lisnic, W J Britt, U Koszinowski, M Messerle, A Krmpotic and S Jonjic. 2010. Recombinant mouse cytomegalovirus expressing a ligand for the NKG2D receptor is attenuated and has improved vaccine properties. J. Clin. Invest. 120: 4532-4545).

The expression of MIC proteins is also induced on many tumor cells where their presence can render them sensitive to targeting and lysis by NK cells. Not surprisingly, a failure to express MIC protein constitutes one means by which many malignant cells, such as those of lung cancer and glioblastoma brain cancer, can escape the innate immune system (Busche, A et al. 2006, NK cell mediated rejection of experimental human lung cancer by genetic over expression of MHC class I chain-related gene A. Human Gene Therapy 17: 135-146; Doubrovina, E S, M M Doubrovin, E Vider, R B Sisson, R J O'Reilly, B Dupont, and Y M Vyas, 2003. Evasion from NK Cell Immunity by MHC Class I Chain-Related Molecules Expressing Colon Adenocarcinoma (2003) J. Immunology 6891-99; Friese, M. et al. 2003. MICA/NKG2D-mediated immunogene therapy of experimental gliomas. Cancer Research 63: 8996-9006; Fuertes, M B, M V Girart, L L Molinero, C I Domaica, L E Rossi, M M Barrio, J Mordoh, GA Rabinovich and NW Zwirner. (2008) Intracellular Retention of the NKG2D Ligand MHC Class I Chain-Related Gene A in Human Melanomas Confers Immune Privilege and Prevents NK Cell-Mediated Cytotoxicity. J. Immunology, 180: 4606-4614).

The high resolution structure of human MICA bound to NKG2D has been solved and demonstrates that the α3 domain of MICA has no direct interaction with the NKG2D (Li et al. 2001. Complex structure of the activating immunoreceptor NKG2D and its MHC class I-like ligand MICA. Nature Immunol. 2: 443-451; Protein Data Bank accession code 1HYR). The α3 domain of MICA, like that of MICB, is connected to the α1-α2 platform domain by a short, flexible linker peptide, and itself is positioned naturally as “spacer” between the platform and the surface of the MIC expressing cell. The three-dimensional structures of the human MICA and MICB α3 domains are nearly identical (root-mean square distance<1 Å on 94 C-αα's) and functionally interchangeable (Holmes et al. 2001. Structural Studies of Allelic Diversity of the MHC Class I Homolog MICB, a Stress-Inducible Ligand for the Activating Immunoreceptor NKG2D. J Immunol. 169: 1395-1400).

Certain non-natural α1-α2 domains of NKG2D ligands modified to bind natural human NKG2D receptors with higher affinities than do natural α1-α2 domains have been described (Candice S. E. Lengyel, Lindsey J. Willis, Patrick Mann, David Baker, Tanja Kortemme, Roland K. Strong and Benjamin J. McFarland. Mutations Designed to Destabilize the Receptor-Bound Conformation Increase MICA-NKG2D Association Rate and Affinity. Journal of Biological Chemistry Vol. 282, no. 42, pp. 30658-30666, 2007; Samuel H. Henager, Melissa A. Hale, Nicholas J. Maurice, Erin C. Dunnington, Carter J. Swanson, Megan J. Peterson, Joseph J. Ban, David J. Culpepper, Luke D. Davies, Lisa K. Sanders, and Benjamin J. McFarland. Combining different design strategies for rational affinity maturation of the MICA-NKG2D interface. Protein Science 2012 VOL 21:1396-1402. Herein we describe non-natural α1-α2 domains of NKG2D ligands that have been modified to bind non-natural NKG2D receptors, themselves mutated at two specific sites, each of which alone results in compromised or loss of binding to all currently known natural α1-α2 domains of human NKG2D ligands (David J. Culpepper, Michael K. Maddox, Andrew B. Caldwell, and Benjamin J. McFarland. Systematic mutation and thermodynamic analysis of central tyrosine pairs in polyspecific NKG2D receptor interactions. (Mol Immunol. 2011 January; 48(4): 516-523).

The instant invention created bispecific molecules comprised of (a) the specifically modified non-natural α1-α2 domains and (b) specifically targeting heterologous molecules, including but not limited to heterologous peptides or polypeptides. The bispecific molecules can bind Chimeric Antigen Receptors (CARs) wherein the Receptor of the CAR is comprised of the non-natural NKG2D receptor ectodomain that binds its cognate modified α1-α2 domains with greater affinity than it does natural α1-α2 domains. The second specificity of the bispecific molecule is comprised of a heterologous component that binds specifically its own respective target molecule. Genetically engineered cells of the immunity system comprised of such CARs and the cognate bispecific molecules can then overcome many of the disadvantages, including known severe systemic toxicities, antigen escape, and limited and uncontrolled persistence of current CAR-T and CAR-NK cell therapeutics (Kalos M, Levine, BL, Porter, DL, Katz, S, Grupp, SA, Bagg, A and June, C. T Cells with chimeric antigen receptors have potent antitumor effects and can establish memory in patients with advanced leukemia. Sci Transl Med 2011; 3:95ra73; Morgan R A, Yang J C, Kitano M, Dudley M E, Laurencot C M, Rosenberg S A. Case report of a serious adverse event following the administration of T cells transduced with a chimeric antigen receptor recognizing ERBB2. Mol Ther 2010, 18:843-851).

T cells and NK-cells can be modified using gene transfer technologies to directly and stably express on their surface transmembrane signaling receptors that confer novel antigen specificities on the T cell by virtue of binding domains taken from antibodies (Saar Gill & Carl H. June. Going viral: chimeric antigen receptor T cell therapy for hematological malignancies. Immunological Reviews 2015. Vol. 263: 68-89; Wolfgang Glienke, Ruth Esser, Christoph Priesner, Julia D. Suerth, Axel Schambach, Winfried S. Wels, Manuel Grez, Stephan Kloess, Lubomir Arseniev and Ulrike Koehl. 2015. Advantages and applications of CAR-ζ expressing natural killer cells. Front. Pharmacol. doi: 10.3389/fphar.2015.00021). Such chimeric antigen receptor-expressing T cells (CAR-T cells) are applications of this approach that combines an antigen recognition domain of a specific antibody with an intracellular domain of the CD3-chain, which is the primary transmitter of signals from endogenous T cell antigen Receptors (TCRs), into a single chimeric protein along with a cytoplasmic polypeptide sequence from a co-stimulatory molecule such as CD27, CD28, ICOS, 4-1BB, or OX40. CARs so constructed can trigger T cell activation upon binding the targeted antigen in a manner similar to an endogenous T cell receptor but independent of the major histocompatibility complex (MHC).

As used herein, a “soluble MIC protein”, “soluble MICA” and “soluble MICB” refer to a MIC protein containing the α1, α2, and α3 domains of the MIC protein but without the transmembrane or intracellular domains. The NKG2D ligands, ULBP1-6, do not naturally possess an α3 domain (Cerwenka A, Lanier L L. 2004. NKG2D ligands: unconventional MHC class I-like molecules exploited by viruses and cancer. Tissue Antigens 61 (5): 335-43. doi:10.1034/j.1399-0039.2003.00070.x. PMID 12753652). An “α1-α2 domain” of an NKG2D ligand refers to the protein domain of the ligand that binds an NKG2D receptor.

In some embodiments, the α1-α2 domains of the non-natural NKG2D ligand proteins of the invention are at least 80% identical or homologous to the native or natural α1-α2 domain of an NKG2D ligand (SEQ ID NOs: 1-9 for MICA, MICB, ULBP1, ULBP2, ULBP3, ULBP4, ULBP5, ULBP6, and OMCP, respectively). In other embodiments, the modified α1-α2 domain is at least 85% identical to a native or natural α1-α2 domain of an NKG2D ligand. In yet other embodiments, the modified α1-α2 domain is at least 90% identical to a native or natural α1-α2 domain of a natural NKG2D ligand protein and binds non-natural NKG2D.

Preferably the modified or non-natural α1-α2 domains of the non-natural MIC proteins of the invention are at least 80% identical or homologous to the native or natural α1-α2 domain of OMPC (SEQ ID NO.:9) or to one of the eight known human NKG2D ligand proteins (SEQ ID NOs: 1-8) and bind the non-natural NKG2D ectodomain. In some embodiments, the non-natural α1-α2 domain is at least 85% identical to a native or natural α1-α2 domain of an NKG2D ligand protein and binds the non-natural NKG2D. In other embodiments, the non-natural α1-α2 platform domain is at least 90%, 95%, 96%, 97%, 98%, or 99% identical to a native or natural α1-α2 platform of a human natural α1-α2 domain protein and binds the non-natural NKG2D.

In some embodiments, a heterologous molecular tag may be fused to the N-terminus or C-terminus of a non-natural α1-α2 domain of a soluble MIC protein to aid in the purification of the soluble MIC protein. Tag sequences include peptides such as a poly-histidine, myc-peptide, a FLAG tag, streptavidin-like tag, or a small molecule such as biotin. Such tags may be removed after isolation of the MIC molecule by methods known to one skilled in the art.

In other embodiments of the invention, specific mutations in α1-α2 domains of NKG2D ligands can be made to create non-natural α1-α2 domains that bind non-natural NKG2D receptors, themselves engineered so as to have reduced affinity for natural NKG2D ligands. This can be done, for example, through genetic engineering. A non-natural NKG2D receptor so modified can be used to create on the surface of NK-cells, T cells, or other cells of the immune system an NKG2D-based Chimeric Antigen Receptor (CAR) that can preferentially bind to and be activated by molecules comprised of the invented non-natural α1-α2 domains. These pairs of non-natural NKG2D receptors and their invented cognate non-natural NKG2D ligands will provide important safety, efficacy, and manufacturing advantages for treating cancer and viral infections as compared to the current CAR-T cells and CAR-NK cells, as described below.

Engineering T cells with CARs has emerged as a promising approach to adoptive T cell therapy for cancer, and CARs targeting many different molecules have been tested in CAR-T cells as therapeutics for malignancies (Porter D L, Levine B L, Kalos M, Bagg A, June C H., 2011. Chimeric antigen receptor-modified T cells in chronic lymphoid leukemia. N Engl J Med. 365:725-733.). While remarkable clinical efficacy has been observed in hundreds of patients receiving adoptive transfer of T cells expressing CD19-specific chimeric antigen receptors, the processes of custom engineering a CAR to target a specific antigen, isolating autologous T cells from the patient, genetically engineering the autologous T cells to express the personalized CAR, expanding the modified cells in vitro, and controlling the quality their production have all been onerous and expensive.

Once the autologous CAR-T cells are infused back into the donor patient, their expansion in vivo cannot be controlled, creating what has been dubbed a “living therapy” which does not demonstrate a dose-response relationship with efficacy (Gill & June, 2015). Furthermore, tumor escape from the CAR-T cell can occur through antigen loss on tumor cells (Stephan A. Grupp, M.D., Ph.D., Michael Kalos, Ph.D., David Barrett, M.D., Ph.D., Richard Aplenc, M.D., Ph.D., David L. Porter, M.D., Susan R. Rheingold, M.D., David T. Teachey, M.D., Anne Chew, Ph.D., Bernd Hauck, Ph.D., J. Fraser Wright, Ph.D., Michael C. Milone, M.D., Ph.D., Bruce L. Levine, Ph.D., and Carl H. June, M.D. Chimeric Antigen Receptor-Modified T Cells for Acute Lymphoid Leukemia. N Engl J Med 2013; 368:1509-1518). While this escape pathway can be addressed by sequential therapy with a differently targeted CAR-T cell or by an initial infusion of a T cell product containing CARs of two or more specificities, such steps represent a significant additional expansion in the manufacturing processes, quality control, and costs.

In addition to CAR-T cells targeting tumors with single chain antibody binding domains (scFv), CAR-T cells that employ the ligand-binding domain of the NKG2D receptor have been studied in animals and recently in humans (Sentman C L, Meehan K R. NKG2D CARs as cell therapy for cancer. Cancer J. 2014 March-April; 20(2):156-9. doi: 10.1097/PPO.0000000000000029; Manfred Lehner, Gabriel Götz, Julia Proff, Niels Schaft, Jan Dörrie, Florian Full, Armin Ensser, Yves A. Muller, Adelheid Cerwenka, Hinrich Abken, Ornella Parolini, Peter F. Ambros, Heinrich Kovar, Wolfgang Holter. Redirecting T Cells to Ewing's Sarcoma Family of Tumors by a Chimeric NKG2D Receptor Expressed by Lentiviral Transduction or mRNA Transfection. Research Article published 15 Feb. 2012|PLOS ONE 10.1371/journal.pone.0031210; www.clinicaltrials.gov NCT02203825). Since NKG2D ligand expression is increased on the surface of stressed cells, such as tumor cells, this family of natural NKG2D ligands is of significant interest as targets for cancer immunotherapies (Spear P, Wu M R, Sentman M L, Sentman C L. NKG2D ligands as therapeutic targets. Cancer Immun. 2013 May 1; 13:8.; Song D G, Ye Q, Santoro S, Fang C, Best A, Powell D J Jr., Chimeric NKG2D CAR-expressing T cell-mediated attack of human ovarian cancer is enhanced by histone deacetylase inhibition. Hum Gene Ther. 2013 March; 24(3):295-305). One NKG2D CAR was a fusion of the full-length NKG2D receptor and CD3ζ (NKG2D); another was with only the ectodomain of NKG2D fused to a second-generation CAR scaffold composed of transmembrane and intracellular domains from CD28 and the signaling domain of CD3ζ (NKG2D28ζ). Since activation of NKG2D is dependent upon the presence of DAP10, a CAR-T cell was also constructed wherein DAP10 was co-expressed with NKG2Dζ (NKG2Dζ10). T cells expressing any of the above NKG2D CARs produced IFNγ and TNFα in response to NKG2D ligand stimulation and in vitro efficiently killed tumor targets expressing NKG2D ligands (Heather VanSeggelen, Joanne A. Hammill, Anna Dvorkin-Gheva, Daniela G. M. Tantalo, Jacek M. Kwiecien, Galina F. Denisova, Brian Rabinovich, Yonghong Wan, Jonathan L. Bramson, T cells engineered with chimeric antigen receptors targeting NKG2D ligands display lethal toxicity in mice, Molecular Therapy 2015 October; 23(20):1600-10; doi:10.1038/mt.2015.119). The cytotoxic potential of NK cells against a wide spectrum of tumor subtypes could also be markedly enhanced by expression of a CAR based on NKG2D-DAP10-CD3r (Yu-Hsiang Chang, John Connolly, Noriko Shimasaki, Kousaku Mimura, Koji Kono, and Dario Campana. Chimeric Receptor with NKG2D Specificity Enhances Natural Killer Cell Activation and Killing of Tumor Cells. Cancer Res; 73(6) Mar. 15, 2013).

However, following infusion into syngeneic murine hosts, significant toxicity occurred with these CAR-T constructs that bind and are activated by natural ligands of the natural NKG2D receptor. Signs of toxicity, including poor body condition, hunched posture, labored breathing, and decreased core body temperature were observed in tumor-bearing and tumor-free mice treated with NKG2D-based CAR-T cells as compared to untreated control mice. The severity of NKG2D CAR-T cell toxicity varied, with NKG2Dζ10 being severely toxic, NKG2D28ζ showing intermediate toxicity, and NKG2Dζ being tolerable. Clinical symptoms of toxicity and mortality rates were exacerbated when mice received chemotherapy prior to adoptive transfer of T cells expressing any of the NKG2D CARs (VanSeggelen et al. 2015). Chemotherapy and radiation are known to induce NKG2D ligands on otherwise healthy tissues (Xiulong Xu, Geetha S Rao, Veronika Groh, Thomas Spies, Paolo Gattuso, Howard L Kaufman, Janet Plate and Richard A Prinz. Major histocompatibility complex class I-related chain A/B (MICA/B) expression in tumor tissue and serum of pancreatic cancer: Role of uric acid accumulation in gemcitabine-induced MICA/B expression. BMC Cancer 2011, 11:194 doi:10.1186/1471-2407-11-194; Gannagé M, Buzyn A, Bogiatzi S I, Lambert M, Soumelis V, Dal Cortivo L, Cavazzana-Calvo M, Brousse N, Caillat-Zucman Induction of NKG2D ligands by gamma radiation and tumor necrosis factor-alpha may participate in the tissue damage during acute graft-versus-host disease. Transplantation. 2008 Mar. 27; 85(6):911-5. doi: 10.1097/TP.0b013e31816691ef.). Further characterization revealed that the toxicity coincided with a systemic cytokine storm and lethal levels of inflammation within the lungs. These data warn that extreme caution must be taken when using natural NKG2D ligands for targeted immunotherapy and demonstrate that T cell expression of CARs targeting such ligands—especially strongly activating CARs—can be detrimental in vivo (VanSeggelen et al. 2015).

CAR-T or CAR-NK cells comprised of ectodomains of non-natural NKG2D receptors that do not or only poorly bind natural NKG2D ligands will not be subject to the above form of activation and thus will not be as toxigenic as cells expressing a CAR based on a natural NKG2D receptor. Furthermore, ectodomains of non-natural NKG2D receptors on cells will not be subject to down-regulation by natural NKG2D ligands in a soluble format or on Myeloid Derived Suppressor Cells (MDSC) (Deng W, Gowen B G, Zhang L, Wang L, Lau S, Iannello A, Xu J, Rovis T L, Xiong N, Raulet D H, 2015. Antitumor immunity. A shed NKG2D ligand that promotes natural killer cell activation and tumor rejection. Science. 2015 Apr. 3; 348(6230):136-9. doi: 10.1126/science.1258867. Epub 2015 Mar. 5). However, when such CAR cells bearing ectodomains of the non-natural NKG2D receptors are engaged by bispecific molecules with the cognate non-natural α1-α2 domains of the instant invention and its heterologous targeting motif which has found and bound its intended target, the CAR will be activated and the CAR-cell's effector functions expressed.

Because the cytokine release and cytolytic activities of CAR-T or CAR-NK cells comprised of non-natural NKG2D receptor ectodomains are not initiated except in the presence of an engaged bispecific molecule comprised of a cognate non-natural α1-α2 domain wherein the bispecific molecule has also engaged an array of its target, their activation can be controlled by the administered bispecific molecules, which as biopharmaceuticals will exhibit pharmacokinetics and pharmacodynamics well known in the field. In the event that an adverse event develops, the physician can simply modify the dosing regimen of the administered bispecific molecule rather than having to deploy an induced suicide mechanism to destroy the infused CAR cells as currently done (Monica Casucci and Attilio Bondanza. Suicide Gene Therapy to Increase the Safety of Chimeric Antigen Receptor-Redirected T Lymphocytes. J Cancer. 2011; 2: 378-382). Furthermore, such bispecific molecules with different specific targeting motifs can be administered simultaneously or sequentially to help address tumor resistance and escape as a results of target antigen loss without having to create, expand and infuse multiple different autologous CAR cells (Gill & June, 2015). Since all CAR constructions can be identical for all CAR cells and the targeting specificity determined simply by the targeting motif of the administered bispecific molecule of the instant invention, CAR-cell manufacturing processes will be simplified and less expensive.

Examples of parent or recipient proteins or polypeptides that are candidates for attachment to non-natural α1-α2 domains of NKG2D ligands include but are not limited to antibodies, proteins comprised of Ig folds or Ig domains, including modified Fc domains that recruit natural molecules or fail to recruit or bind natural molecules, globulins, albumens, fibronectins and fibronectin domains, integrins, fluorescent proteins, enzymes, outer membrane proteins, receptor proteins, T cell receptors, chimeric antigen receptors, viral antigens, virus capsids, viral ligands for cell receptors, histones, hormones, cytokines and modified cytokines such as interleukins, knottins, cyclic peptides or polypeptides, major histocompatibility (MHC) family proteins, MIC proteins, lectins, and ligands for lectins. It is also possible to attach non-protein molecules such a polysaccharides, dendrimers, polyglycols, peptidoglycans, antibiotics, and polyketides to the modified α1-α2 domains of NKG2D ligands.

Thus, the instant invention expands the diversity and practicality of this remarkable, very promising immunologic approach to managing cancer with CAR-T cells, CAR-NK cells, and CAR-macrophage-like cells while overcoming many of these current, recognized difficulties.

As used herein “peptide”, “polypeptide”, and “protein” are used interchangeably; and a “heterologous molecule”, “heterologous peptide”, “heterologous sequence” or “heterologous atom” is a molecule, peptide, nucleic acid or amino acid sequence, or atom, respectively, that is not naturally or normally found in physical conjunction with the subject molecule. As used herein, “non-natural” and “modified” are used interchangeably. As used herein, “natural”, “native”, and “wild-type” are used interchangeably and “NKG2D” and “NKG2D receptor” are used interchangeably. The term “antibody” herein is used in the broadest sense and specifically covers monoclonal antibodies, multispecific antibodies (e.g. bispecific antibodies), and antibody fragments, so long as they exhibit the desired biological activity. “Antibody fragments” comprise a portion of an antibody, preferably comprising the antigen binding region thereof. Examples of antibody fragments include Fab, Fab′, F(ab′)₂, Fv fragments and insertible Fv's; diabodies; linear antibodies; single-chain antibody molecules; and multi-specific antibodies formed from antibody fragment(s). “Antibody fragments” can also comprise the Fc portion of an antibody.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

All references cited herein are hereby incorporated by reference in their entireties, whether previously specifically incorporated or not. As used herein, the terms “a”, “an”, and “any” are each intended to include both the singular and plural forms.

Having now fully described the invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and may be applied to the essential features hereinbefore set forth.

### EXAMPLES

### Modified NKG2D Receptor Ectodomain and Modified α1-α2 Domains of NKG2D Ligands

### Example 1. Modification of Tyrosine 152 to Alanine (Y152A) and Tyrosine 199 to Phenylalanine (Y199F) of the Human NKG2D Receptor to Create an Inert NKG2D Ectodomain

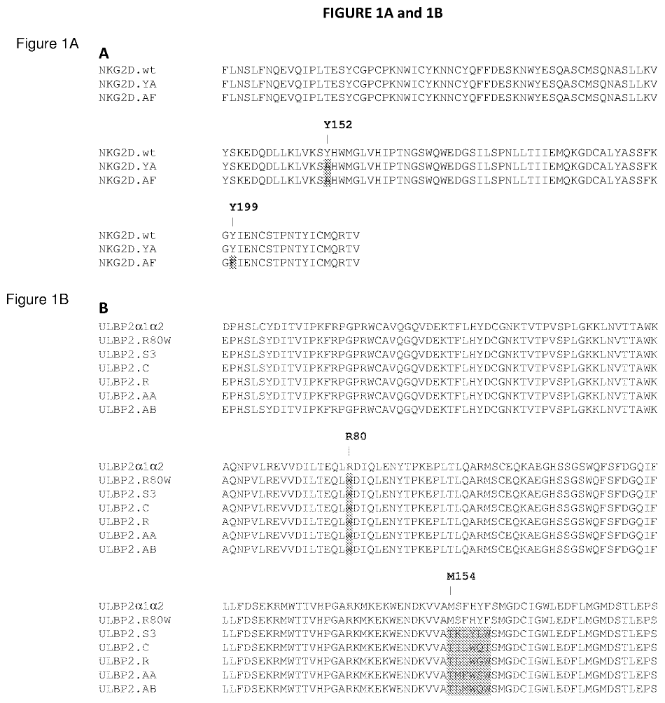

It had been demonstrated by others that mutations at tyrosine 152 or at tyrosine 199 in human NKG2D, the equivalent of positions 75 and 122 of the NKG2D ectodomain (FIG. 1A, SEQ ID NO.:17) can greatly reduce binding to the natural ligand, MICA (David J. Culpepper, Michael K. Maddox, Andrew B. Caldwell, and Benjamin J. McFarland. Systematic mutation and thermodynamic analysis of central tyrosine pairs in polyspecific NKG2D receptor interactions. Mol Immunol. 2011 January; 48(4): 516-523). We reasoned that while mutation of either tyrosine residue greatly affected the ability of NKG2D to bind to its natural ligands, simultaneous mutation at both tyrosine 152 (Y152) and tyrosine 199 (Y199) would virtually eliminate the receptor's ability to engage with all native ligands. We therefore sought to explore individual and combinatorial Y152 and Y199 substitutions and characterize them with regard to their biochemical behavior with the objective of identifying both single and double-mutant variants incapable of engaging any natural ligands. Those variants that also expressed and assembled well were of particular interest as these signified inert ligands that could be more easily produced for analysis.

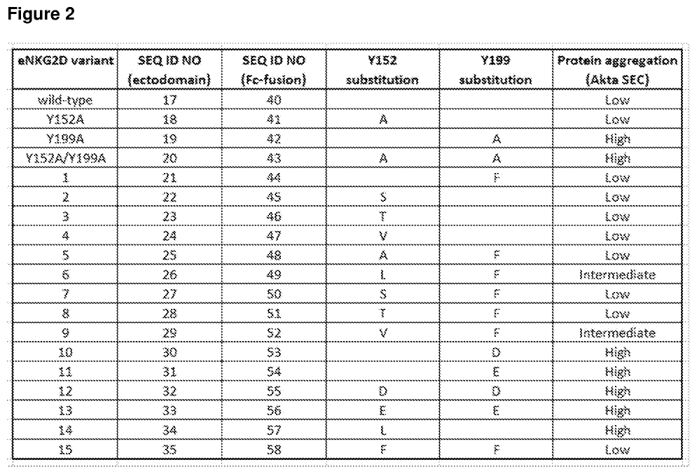

Natural NKG2D (wild-type) ectodomain (NKG2D.wt, SEQ ID NO: 17) and candidate non-natural NKG2D variant ectodomains (SEQ ID NOs: 18-35)—also termed “engineered NKG2D” or “eNKG2D” were cloned as fusions to the C-terminus of human IgG1 Fc (without Fab domains), via a short factor Xa recognizable Ile-Glu-Gly-Arg linker (SEQ ID NO: 38) and are interchangeably referred to as Fc-NKG2D.wt or NKG2D.wt and Fc-eNKG2D or eNKG2D (SEQ ID NOs: 40-58). gBlocks® DNA Fragments (Integrated DNA Technologies, San Diego, CA), corresponding to the MHCI signal sequence (SEQ ID NOs: 36 and 37), human IgG1 Fc with linker (SEQ ID NO: 39), and NKG2D ectodomain variants (SEQ ID NOs: 59-77) were synthesized and inserted into pD2610-V12 (ATUM, Newark, CA). DNA constructs exploring substitutions at Y152, Y199, or a combination of Y152/Y199 mutations (FIG. 2) were expressed transiently in Expi293™ cells (ThermoFisher Scientific, Waltham, MA) and secreted protein purified by Protein A affinity chromatography (cat. no. 20334, Pierce Biotechnology, Rockford, IL). Eluted material was characterized by size-exclusion chromatography (SEC) on Akta Pur Superdex columns and correctly assembled, size-appropriate material was fractionated and isolated from aggregate peaks prior to inclusion in assays.

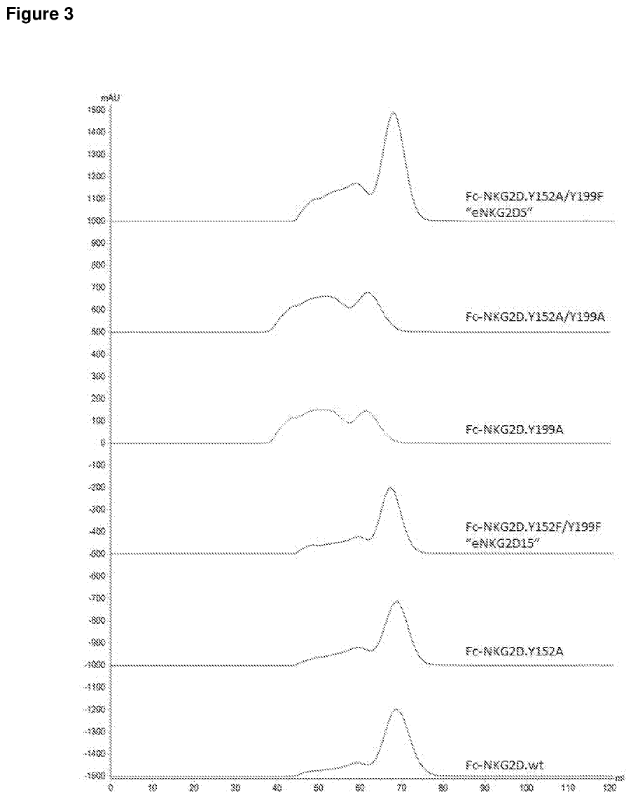

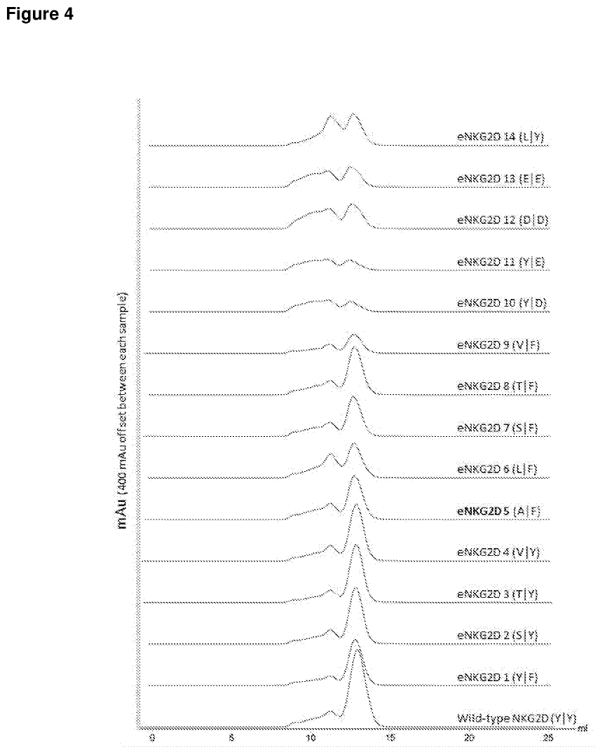

SEC characterization of purified NKG2D.Y199A-Fc fusion revealed a composition of predominantly aggregated material (FIG. 3). In comparison, both the natural Fc-NKG2D fusion and Fc-NKG2D.Y152A fusion material were distinguished by a discrete, non-aggregated peak that was readily differentiated from more rapidly migrating aggregate. The effect of the Y199A mutation on aggregation was also apparent in the Y152A/Y199A double-mutant Fc-NKG2D fusion variant, indicating that it had an overriding influence on protein misfolding (FIG. 3). This aspect of including Y199A with any combination of Y152 mutations in NKG2D variants therefore presented a challenge for the production of material necessary for subsequent engineering efforts and raised a concern about assembly and presentation on a cell surface. As a consequence, an effort was made to explore other substitutions at Y152 and Y199 that could be combined to yield a more robust molecule. eNKG2D combinatorial Y152 and Y199 mutant candidates were examined as Fc fusions and detailed in (FIG. 2). In addition, all purified and expressed Fc-eNKG2D fusion candidates were profiled by SEC and their chromatograms revealed varying levels of aggregate formation (FIGS. 2-4). Of the single amino acid substitutions explored at residue 152 alanine, serine, threonine, and valine all did not impact assembly of the Fc-NKG2D molecule although Y152-leucine (Y152L) resulted in highly aggregated material. Similar to alanine, neither glutamate nor aspartate were tolerated at position 199, although phenylalanine only modestly increased aggregate formation. Of the combinations of mutations that were explored, Y152A/Y199F, Y152S/Y199F, Y152T/Y199F, and Y152F/Y199F did not negatively impact the desired dimer formation, whereas other combinations resulted in increased aggregation (FIGS. 2-4).

### Example 2: Generation of Antibody-Based Bispecific Molecules, “MicAbodies”, with Non-natural NKG2D Ligand Variants

To generate non-natural MicA variants fused to human IgG1, the DNA polynucleotides encoding the α1-α2 domains of, for example, MICwed (SEQ ID NO: 79) and MIC25 (SEQ ID NO: 81), were PCR amplified using primers that also introduced the polynucleotide encoding either an APTSSSGGGGS linker for fusion to C-terminal kappa light chain (SEQ ID NO: 84) or for a GGGS linker for fusion to C-terminal heavy chain of human IgG1 (SEQ ID NO: 82). Furthermore, two mutations were introduced into the CH2 domain of the heavy chain—D265A/N297A (Kabat numbering)—that reduce binding to all FcγR receptors thus eliminating antibody-dependent cell cytotoxicity (ADCC) function (Shields et al., 2001 JBC, 276:6591-6604]. The polynucleotide encoding the α1-α2 domain of wild-type ULBP2 (ULBP2.wt) without its GPI-linkage (SEQ ID NO: 12) was similarly cloned and fused to the DNA polynucleotides encoding the linkers and the IgG1 heavy chain or light chain. These bispecific antibodies—termed “MicAbody™” in the singular, “MicAbodies” in the plural—are bivalent for the fused α1-α2 domain. Examples of antibodies used to generate MicAbodies for the purposes of exploring eNKG2D engineering include but were not limited to trastuzumab (SEQ ID NOs: 94 and 96) and ritixumab (SEQ ID NOs: 98 and 100) and subsequently termed “trastuzumab-MicAbody” (e.g. SEQ ID NOs: 102 and 104) and “rituximab-MicAbody” (e.g. SEQ ID NO: 106), respectively. The fusion constructs were inserted individually into pD2610-V12 (ATUM, Newark, CA) via Gibson cloning (New England Biolabs Inc., Ipswich, MA). For a given antibody recognizing a specific antigen, the plasmid encoding the heavy chain and the plasmid encoding the light chain fused to either natural or non-natural NKG2D ligand were co-transfected for transient expression in Expi293™ cells (ThermoFisher Scientific, Waltham, MA). Alternatively, the plasmid encoding the heavy chain fused to either natural or non-natural NKG2D ligand and the plasmid for light chain were co-transfected. Secreted bispecific antibodies were purified by Protein A affinity chromatography (cat. no. 20334, Pierce Biotechnology, Rockford, IL), eluted material was characterized by size-exclusion chromatography (SEC) on Akta Pur Superdex columns, and fractionation performed as needed. In addition, SDS-PAGE analysis was performed on purified samples to verify the expected molecular weights of the fused heavy chain and fused light chain species.

### Example 3: Identifying Modified NK2GD Variants Incapable of Binding to Either Natural NKG2D-binding Ligands or to Non-natural Ligands that have Enhanced Binding to Wild-type NKG2D

The binding affinities of α1-α2 variants to the extracellular domains of natural (wild-type) NKG2D and non-natural eNKG2D proteins were analyzed using a plate-based ELISA method. Each of the SEC fractionated natural Fc-NKG2D and non-natural Fc-eNKG2D fusions were coated overnight at 4° C. onto separate wells of Nunc Maxisorp 96 well plates (Thermo Fisher Scientific, Waltham, MA) using a coating concentration of 1 μg/mL in phosphate-buffered saline (PBS). The plates were washed three times in PBS/0.05% Tween-20 (PBS-T) at 20-22° C., and blocked with 0.5% bovine serum albumin in PBS (PBS-B) for 2 hours at 20-22° C. MicAbodies were titrated against the plate-bound natural or non-natural Fc-NKG2D fusions for 60 minutes at 20-22° C. in PBS/0.5% bovine serum albumin (BSA)/0.05% Tween-20 (PBS-BT), washed 3 times with PBS-T at 20-22° C., and the bound bispecific proteins detected using an HRP-conjugated anti-human kappa in PBS-BT (Abcam, Cambridge MA) and developed with 1-Step™ Ultra TMB ELISA Substrate Solution (Thermo Fisher Scientific, Waltham, MA). The binding of the ULBP2.wt rituximab-MicAbody (SEQ ID NOs: 98 and 106) discriminated between wild-type NKG2D and eNKG2D variants with reduced binding to the latter, and ligand variants—MICwed (SEQ ID NOs: 96 and 102) and MIC25 (SEQ ID NOs: 96 and 104)—were more stringent at identifying eNKG2D variants with abolished ligand binding. The binding behaviors for each eNKG2D variant against all three bispecific ligands revealed the combinations of NKG2D modifications that led to the greatest reduction in binding of wild-type and variant ligands and enabled the selection of lead inert NKG2D variants.

Additional biophysical analysis of eNKG2D variant binding to ligands was also performed with Bio-Layer Interferometry (BLI) using the FortéBio Octet system (all FortéBio LLC, Fremont, CA). For these experiments human NKG2D ligands MICA-Fc, MICB-Fc, ULBP1-Fc, ULBP2-Fc, ULBP3-Fc, and ULBP4-Fc were purchased from R&D Systems, Inc. (Minneapolis, MN). Ligands in the MicAbody format were captured on anti-human IgG Fc capture (AHC) biosensor tips. After a baselines were established, tips were exposed to a titration series of Fc-eNKG2D fusion proteins ranging from 300 nM to 0.41 nM and association/dissociation kinetics monitored with all steps performed in PBS-BT. Subsequently, Fc-eNKG2D fusion proteins were captured onto AHC tips and MicAbodies were titrated to characterize binding kinetics.

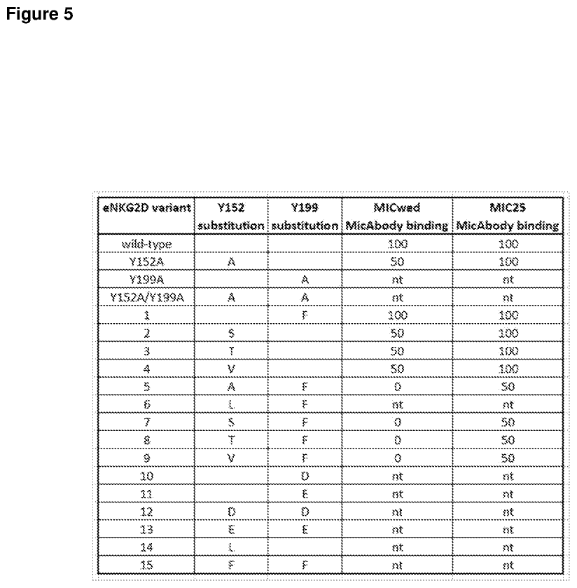

To determine the maximum response as defined by binding of natural NKG2D to either MICwed or MIC25, natural Fc-NKG2D fusions were captured onto AHC biosensors and 20 nM trastuzumab-MICwed or 20 nM trastuzumab-MIC25 MicAbodies were incubated for two minutes and then dissociation kinetics observed for 30 seconds. Binding analysis under the same conditions was then performed with Fc-eNKG2D fusion receptors as the capture agent, and the level of binding for each eNKG2D ranked as a percentage of the maximal binding response established by Fc-NKG2D.wt (FIG. 5). For MICwed, the responses of all single mutant Fc-eNKG2D variants, except for Y199F, were diminished to 50%. Y199F maintained 100% binding response. However, all double-mutant Fc-eNKG2D variants had completely abolished binding to MICwed. For MIC25, all single mutant Fc-eNKG2D variants and Y152V/Y199F maintained 100% binding response relative to wild-type Fc-NKG2D binding. However, binding was reduced to 50% with several of the double-mutant Fc-eNKG2D variants including Y152A/Y199F, Y152S/Y199F, and Y152T/Y199F.

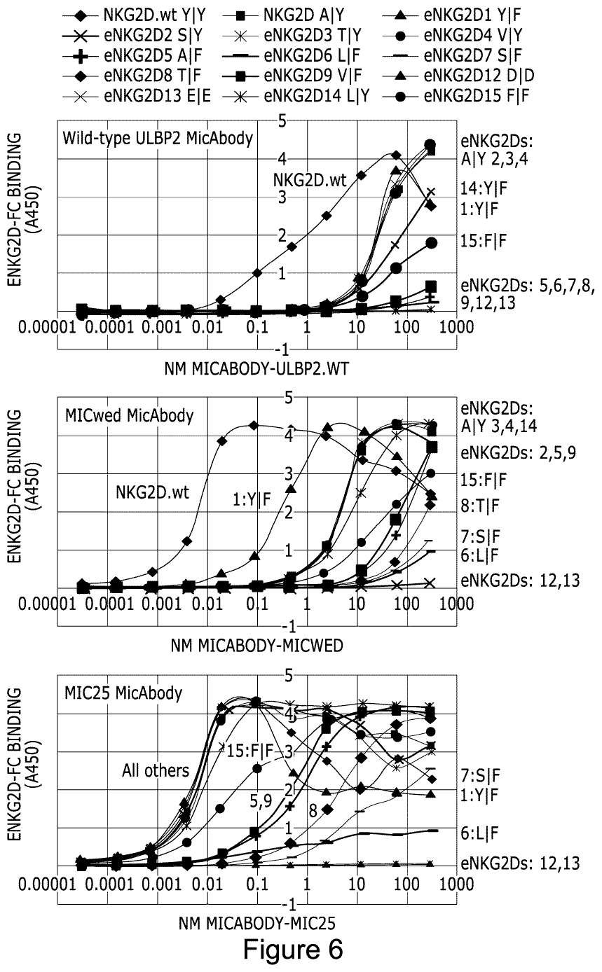

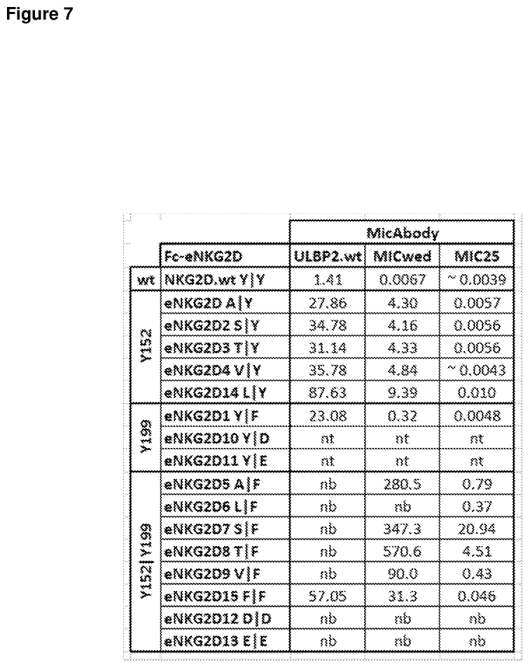

ELISA assays with Fc-eNKG2D fusions as capture agents were performed with ULBP2.wt, MICwed, MIC25 MicAbodies titrated starting at 300 nM (FIG. 6). EC₅₀ values were calculated when possible using GraphPad Prism (FIG. 7). Natural NKG2D bound to ULBP2, MICwed, and MIC25-based MicAbodies with affinities calculated as Kds values of 1.4, 0.007, and 0.005 nM, respectively. While affinity was diminished for ULBP2 and MICwed MicAbodies with all single mutant eNKG2D candidates, binding of MIC25 to eNKG2D candidates was retained. However, all double-mutant eNKG2D candidates had eliminated or significantly reduced binding to all three ligands—ULBP2, MICwed, and MIC25—in Micabody formats.

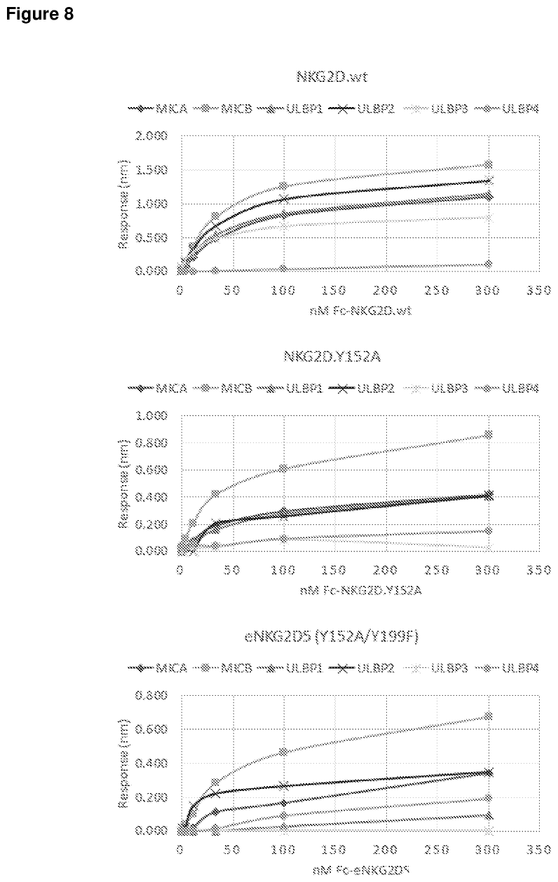

eNKG2D variants eNKG2D5 (Y152A/Y199F), eNKG2D7 (Y152S/Y199F), eNKG2D8 (Y152T/Y199F), and eNKG2D9 (Y152V/Y199F) had reduced or abolished binding to ULBP2, MICwed, and MIC25-based MicAbodies by both Octet analysis and ELISA (FIGS. 5 and 7). Furthermore, eNKG2Ds 5, 7, and 8 had the least amount of aggregation, suggestive of more robust protein assembly upon 293T expression (FIG. 2). eNKG2D5 (SEQ ID NO: 48) was examined more closely for binding to wild-type ligands as MicAbodies captured on Octet AHC tips. Single mutant Fc-NKG2D.Y152A (SEQ ID NO: 41) had reduced binding to all natural ligands relative to natural (SEQ ID NO: 40) NKG2D (FIG. 8). The response curve for binding of eNKG2D5 (Y152A/Y199F) was reduced even further relative to Y152A eNKG2D. eNKG2D5 (Y152A/Y199F, henceforth referred to as “AF” or “NKG2D.AF”) was chosen as the lead NKG2D variant for which to engineer cognate selective, orthogonal, non-natural ligands.

### Example 4: Constructing Orthogonal Non-Natural α1-α2 Domains with Selective Binding to Non-natural NKG2D.AF Ectodomain

We employed phage display to engineer orthogonal non-natural α1-α2 domains that exhibit selective binding to the Fc-NKG2D AF (SEQ ID NO: 48) receptor. As a starting point, the non-natural ULBP2.R80W α1-α2 domain (FIG. 1B; SEQ ID NO: 108) with high affinity for natural, wild-type NKG2D (NKG2D.wt) ectodomain was selected as the parent domain for further mutagenesis and screening by phage display. Synthetic DNA libraries were generated for the α1-α2 domain of ULBP2.R80W (SEQ ID NO: 108) which additionally has a C8S mutation to eliminate the potential for disulfide linkages. Codons of amino acid residues of the ligand that in the bound state are positioned in close proximity to the Y152 and Y199 positions on the natural NKG2D receptor were replaced with NNK codons; the libraries consisted of NNK codons at positions 154-159 (FIG. 1B; SEQ ID NO: 110). Libraries were cloned as fusions to the pIII minor coat protein of M13 phage, and phage particles displaying the mutagenized α1-α2 domain variants were produced in SS320 E. coli cells according to standard methodologies (Andris-Widhopf, J., Steinberger, P., Fuller, R., Rader, C., and Barbas, C. F., 3rd. (2011). These α1-α2 phage display libraries were sorted for high binding affinity to the non-natural NKG2D.AF receptor by selectively capturing phage clones bound to biotinylated Fc-NKG2D.AF protein in the presence of non-biotinylated natural Fc-NKG2D.wt competitor protein. Selective clones were enriched by cycling through multiple rounds of competitive selection with increasing concentrations of the non-biotinylated natural Fc-NKG2D.

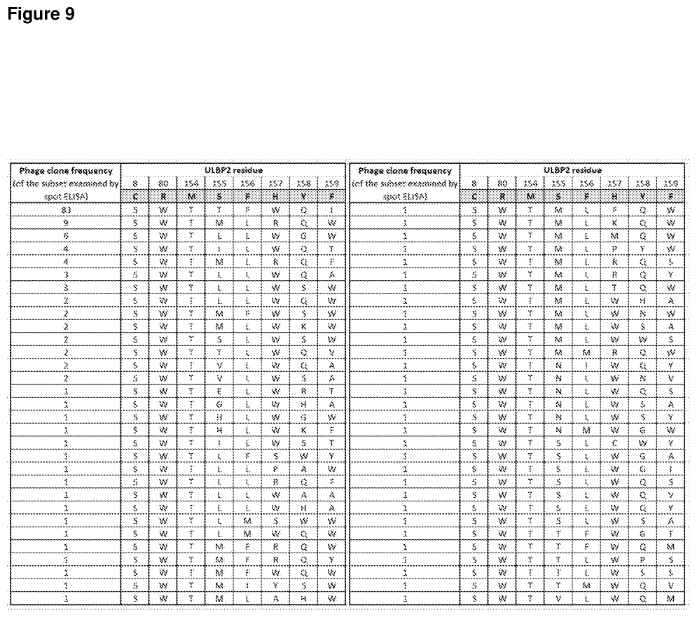

After four rounds of selection, phage clones were individually arrayed in 96-well format, spot ELISAs were performed to verify preferred differential binding to plate-bound non-natural NKG2D.AF versus NKG2D.wt. Bound phages were detected with biotinylated M13 phage coat protein monoclonal antibody E1 (ThermoFisher Scientific, Waltham, MA), streptavidin-HRP detection (R&D Systems, Minneapolis, MN), and 1-Step Ultra TMB ELISA development (ThermoFisher Scientific, Waltham, MA). The spot ELISA signal for each clone was expressed as a ratio of phage binding NKG2D.AF to phage binding NKG2D.wt. Those phages with a ratio greater than or equal to 14 were sequenced to identify the specific mutations within the NNK mutagenized regions. FIG. 9 shows the selected amino acid residues for each α1-α2 phage variant that selectively bound NKG2D.AF. In instances where multiple clones representing the same sequence were identified, the ratio of ELISA signals was plotted, and consistency of phage clones was verified by the clustering of data points (data not shown).

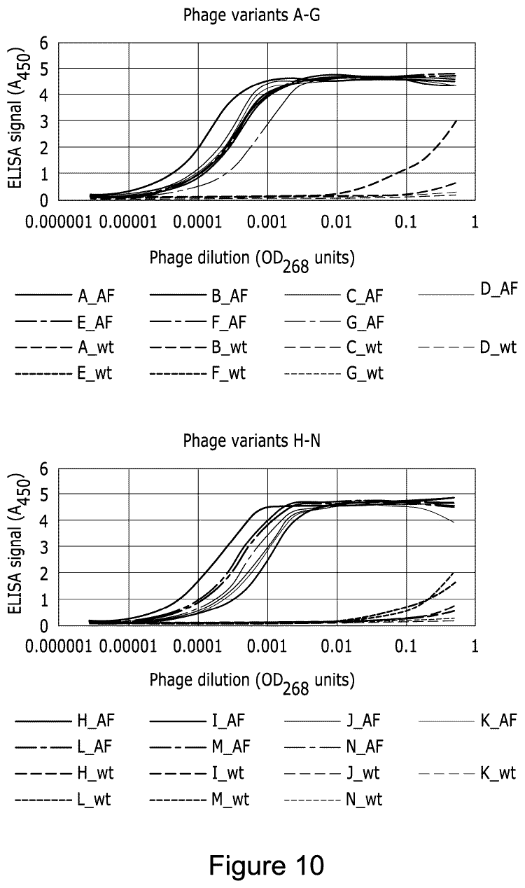

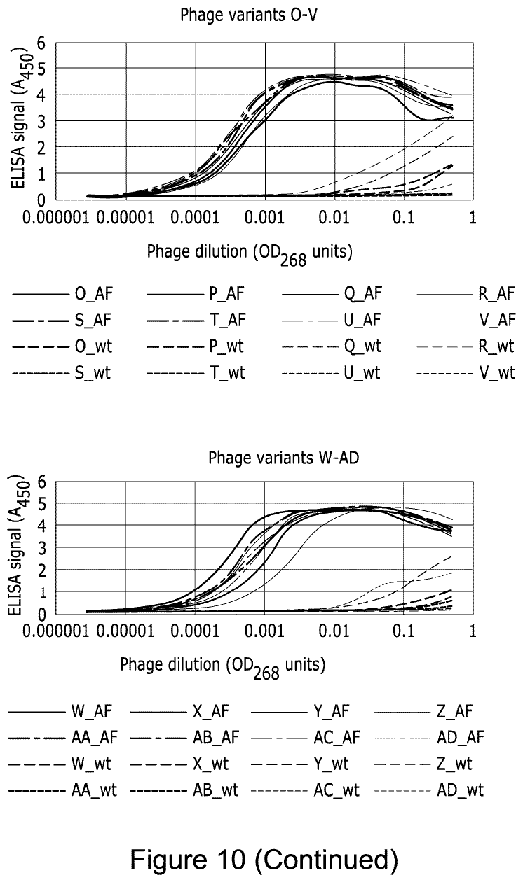

Thirty of the variants identified in ELISAs were expanded in individual monocultures to generate high titer microbatches of phage. Purified phage concentrations were normalized to an OD₂₆₈=0.5 then subject to 1:3 dilution series against plate-bound Fc-NKG2D.AF or Fc-NKG2D.wt with phage detection and ELISA development performed as described above. All thirty variants assayed in this manner consistently demonstrated selective binding to NKG2D.AF with little to no binding to NKG2D.wt (FIG. 10) even at the highest concentrations of phage assayed. The selected phages also exhibited a shift of two or more logs of phage concentration to achieve half-maximal binding between NKG2D.AF and NKG2D.wt.

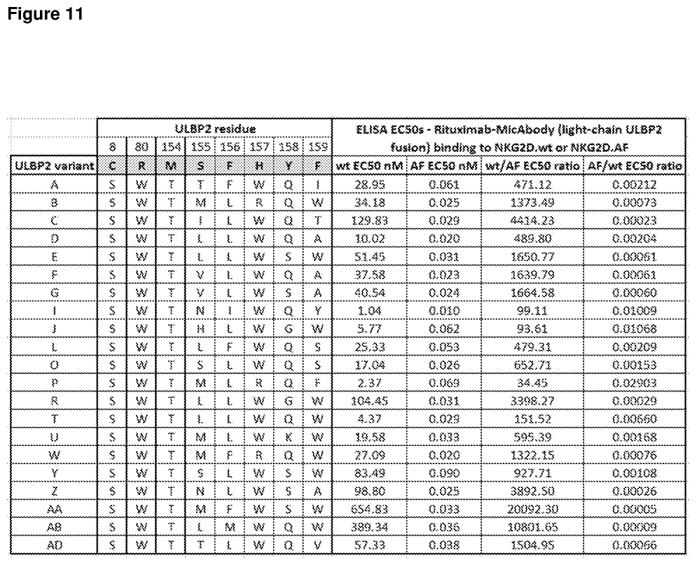

To confirm that the NKG2D.AF-selective α1-α2 domain variants retained specific binding properties in the context of antibody fusions, 21 variants (FIG. 11; e.g. SEQ ID NOs: 111-118) were cloned as C-terminal fusions with an APTSSSGGGGS linker to the light chain of the rituximab antibody (SEQ ID NOs: 119-126). The resulting fusions were cloned into the mammalian expression vector pD2610-V12 (ATUM, Newark, CA) via Gibson cloning (New England Biolabs Inc., Ipswich, MA) and co-expressed with the heavy chain of the parent antibody (SEQ ID NO: 99) as paired full IgG antibodies. Transient expressions were carried out in Expi293™ cells (ThermoFisher Scientific, Waltham, MA) according to the manufacturer's protocol, and purified using standard protein-A affinity chromatography (cat. no. 20334, Pierce Biotechnology, Rockford, IL). ELISAs measuring the binding of each variant ULBP2 α1-α2 antibody fusions to non-natural Fc-NKG2D.AF and to natural Fc-NKG2D.wt demonstrated their significantly greater binding affinity toward NKG2D.AF relative to the natural NKG2D.wt (FIG. 11). Collectively, these data demonstrated the invention of non-natural, orthogonal α1-α2 domains that possessed high affinity binding to the non-natural NKG2D.AF receptor and significantly reduced binding affinity to the natural NKG2D receptor. Furthermore, fusions of these orthogonal α1-α2 domains to antibody polypeptides retained their selective binding properties and were used, for example, in the context of chimeric antigen receptor (CAR) T cells, to redirect non-natural NKG2D.AF receptors toward specific antigens.

### Example 5: Identifying Non-natural NKG2D Ligands that can Discriminate Between Non-natural NKG2D Receptor Variants by Selectively Binding One or the Other

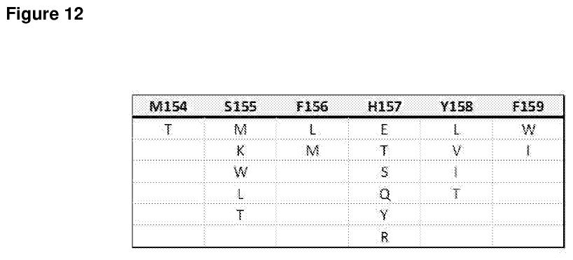

Phage display to engineer orthogonal non-natural α1-α2 domains with selective binding to NKG2D.Y152A (henceforth referred to as NKG2D.YA, receptor was performed with non-natural ULBP2.R80W α1-α2 domain (SEQ ID NO: 108) as the starting point as described above. The α1-α2 phage display libraries were panned for high binding affinity to the non-natural Fc-NKG2D.YA receptor by selectively capturing phage clones bound to biotinylated Fc-NKG2D.YA (SEQ ID NO: 41) protein in the presence of non-biotinylated natural Fc-NKG2D.wt (SEQ ID NO: 40) competitor protein. Additional phage clone validation work resulted in the identification of variants with preferential binding to Fc-NKG2D.YA versus Fc-NKG2D.wt (FIG. 12). ULBP2.S3 (SEQ ID NO: 127), for example, consistently demonstrated selective binding by ELISA and Octet analysis (both in monomeric His-tagged and bispecific antibody fused format) to non-natural NKG2D.YA relative to natural NKG2D.wt. This represented a distinct form of the invention of non-natural orthogonal α1-α2 domains possessing high affinity binding to non-natural NKG2D receptors (in this case NKG2D.YA as opposed to NKG2D.AF as in Example 4). Furthermore, fusions of orthogonal α1-α2 domains to antibody polypeptides retained their selective binding properties and were used to selectively redirect non-natural NKG2D receptors towards specific molecules determined by fused heterologous peptides such as antibodies.

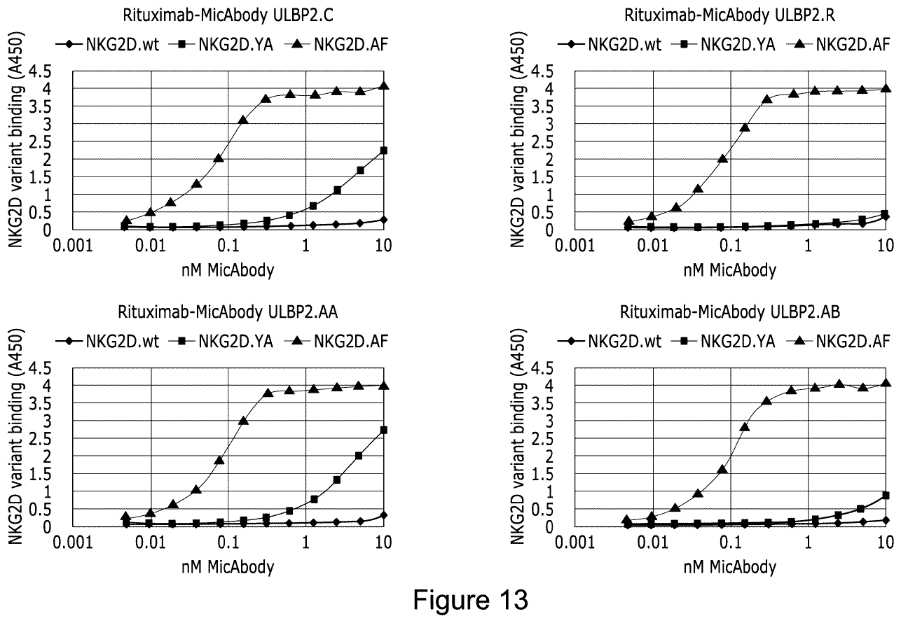

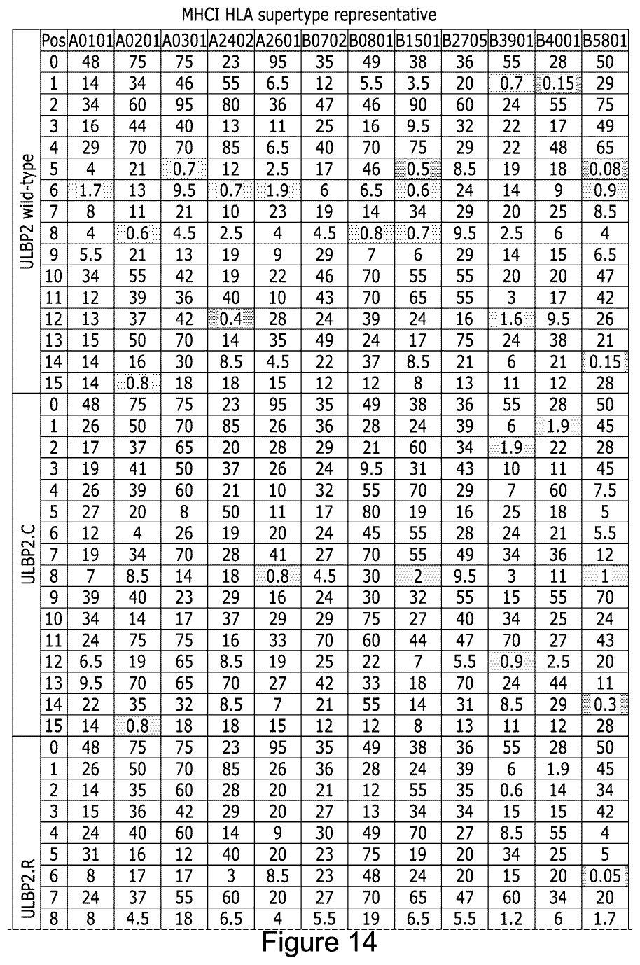

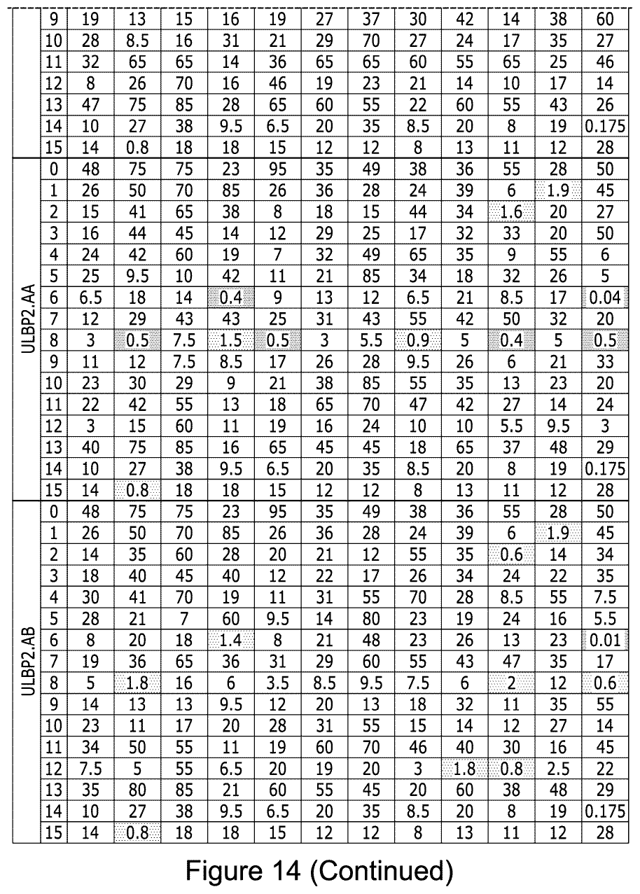

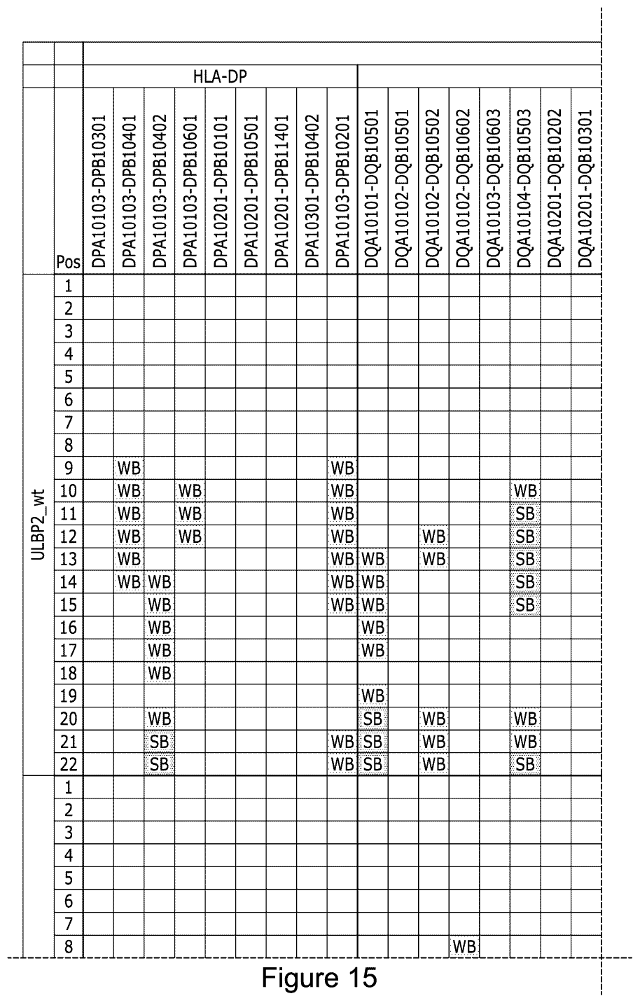

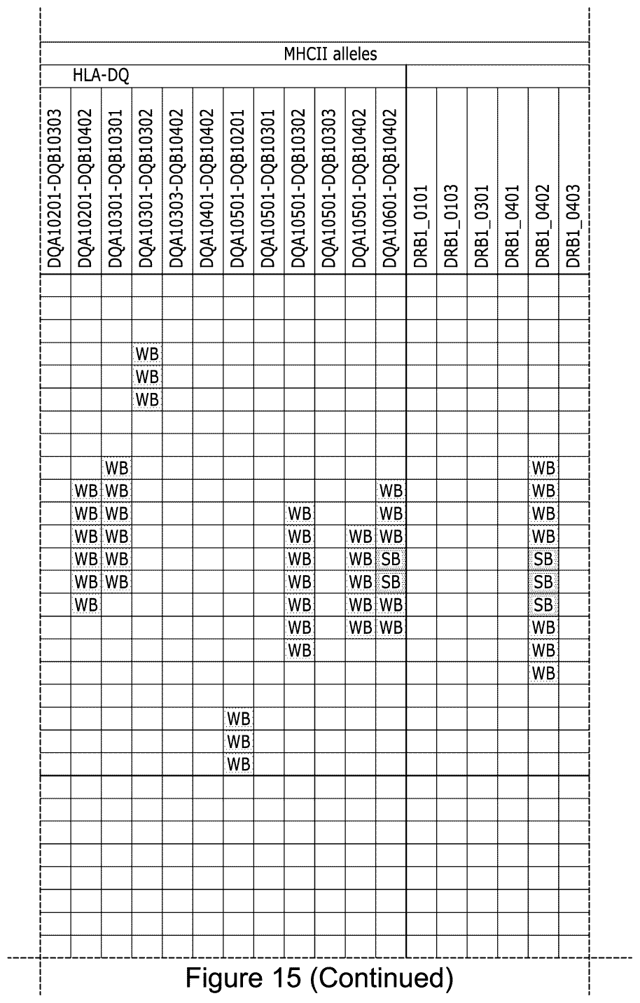

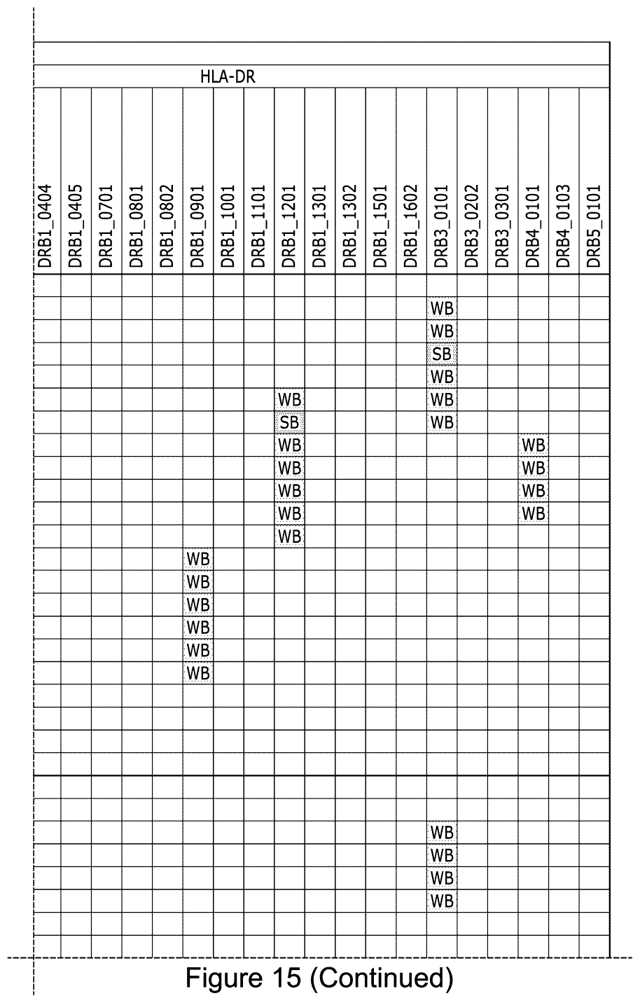

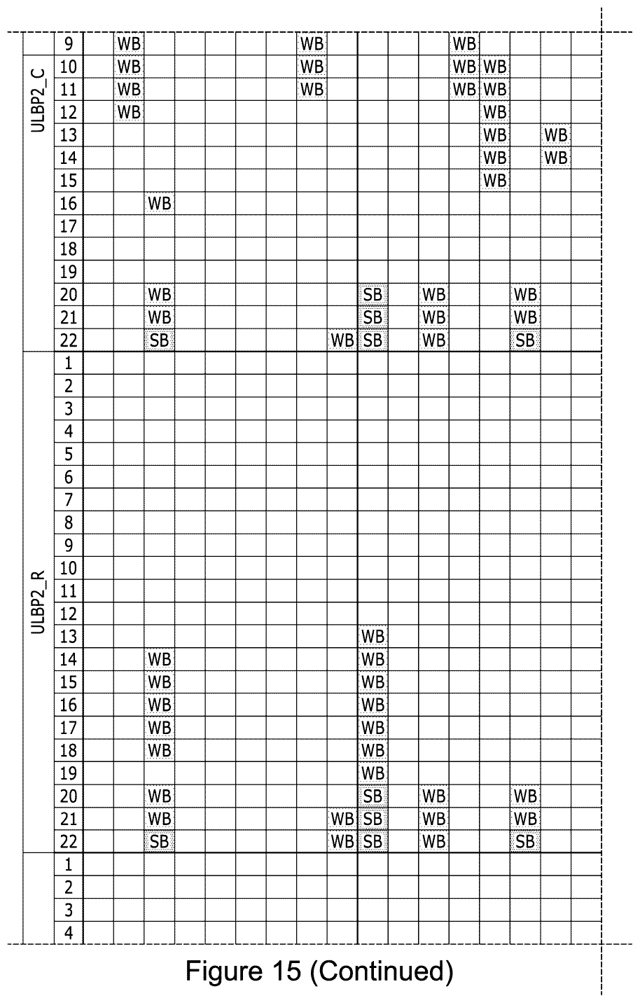

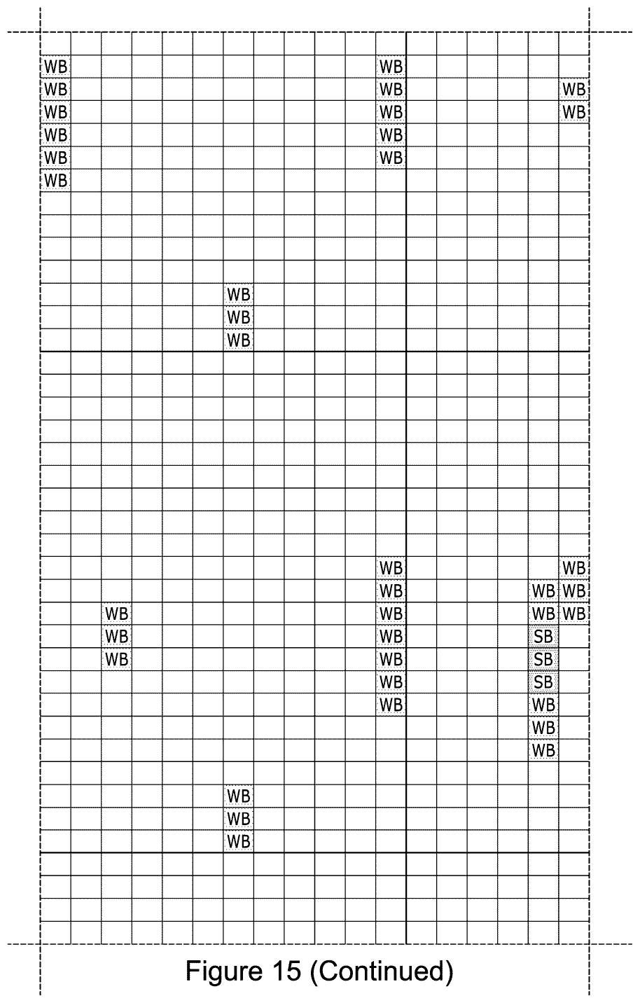

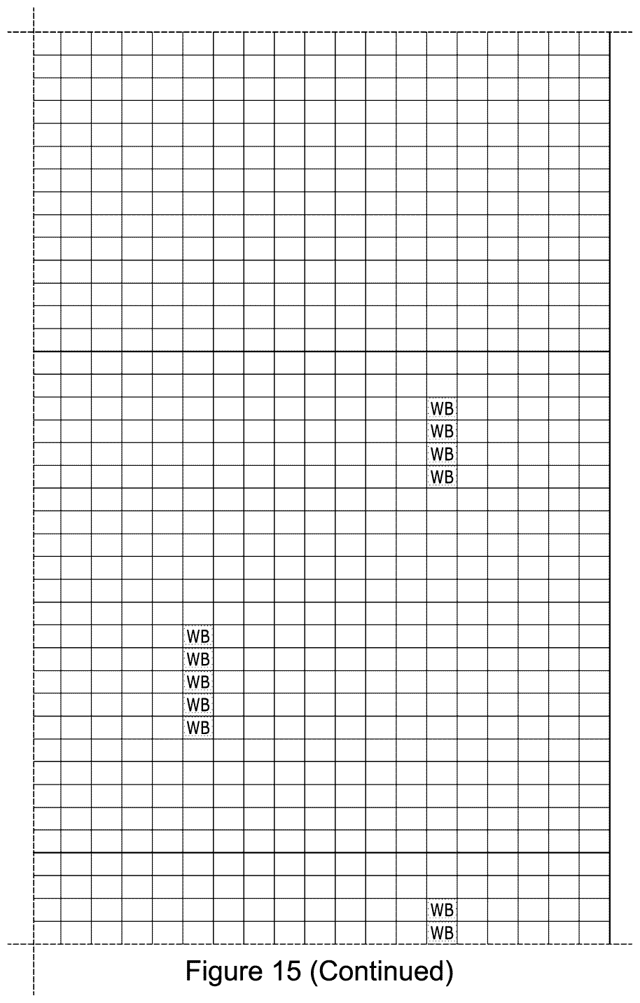

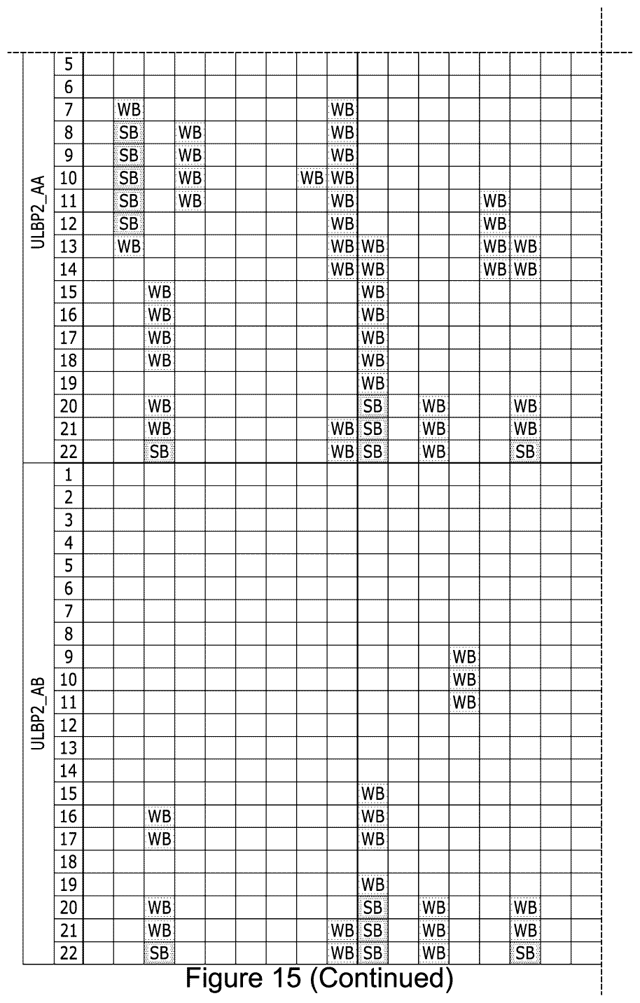

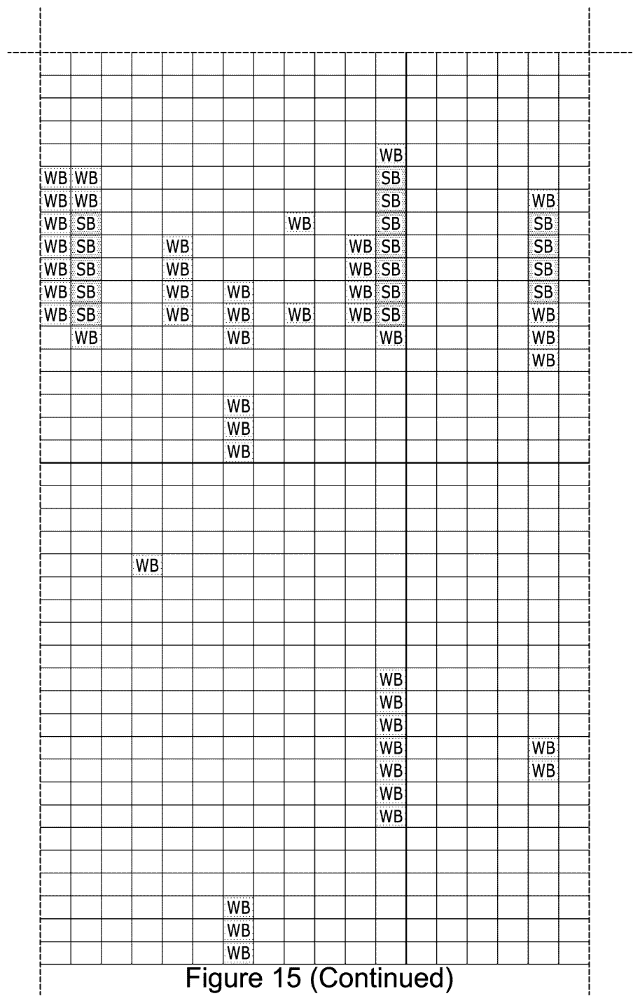

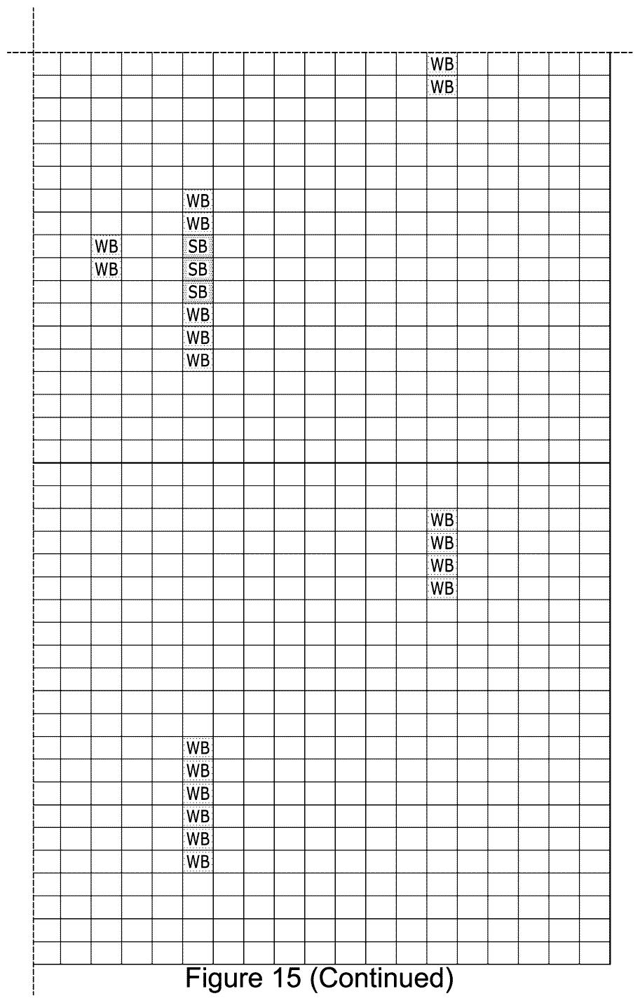

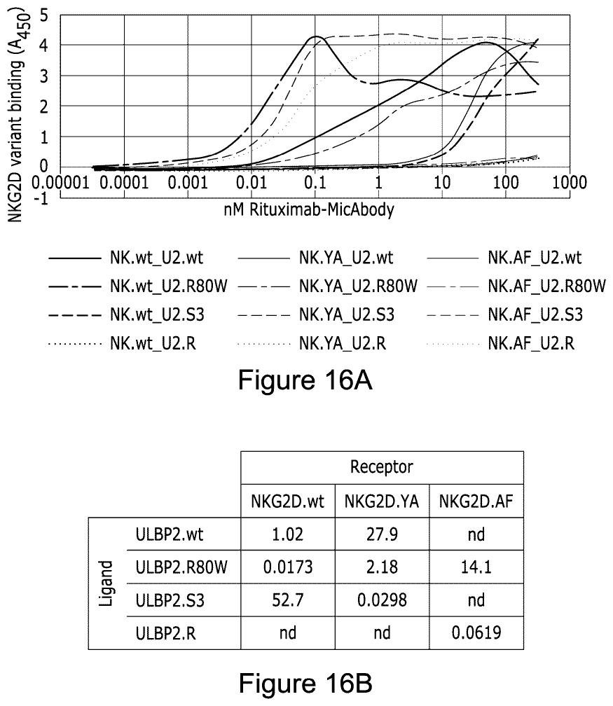

In order to determine whether a non-natural α1-α2 domain with selective binding to NKG2D.YA (ULBP2.S3, SEQ ID NO: 127) and the non-natural α1-α2 domains with selective binding to NKG2D.AF could discriminate between these two non-natural receptor variants, titration ELISAs were performed. All 21 of the selected α1-α2 variants that bound NKG2D.AF were directly compared for binding to NKG2D.AF versus NKG2D.YA. Of these, four demonstrated the properties of inability to bind NKG2D.wt, strong affinity for NKG2D.AF, and greatly reduced (15-20 fold) or eliminated binding to NKG2D.YA relative to NKG2D.AF (FIG. 13). These four non-natural ULBP2 α1-α2 variants—ULBP2.C, ULBP2.R, ULBP2.AA, and ULBP2.AB (SEQ ID NOs: 111, 113, 115, and 117)—were also examined for alterations in predicted immunogenicity profile relative to the wild-type ULBP2 peptide sequence (SEQ ID NO: 4) using the NetMHC4.0 Server (for peptide-MHC class I binding querying against all the HLA supertype representatives with 9-mer peptide analysis; http://www.cbs.dtu.dk/services/NetMHC/) and NetMHCII 2.3 Server (for peptide-MHC class II binding querying against HLA-DR, HLA-DQ, HLA-DP haplotypes with 15-mer peptide analysis; http://www.cbs.dtu.dk/services/NetMHCII/), both algorithms which were developed by the Technical University of Denmark (http://www.bioinformatics.dtu.dk/; Andreatta M and Nielsen M, Gapped sequence alignment using artificial neural networks: application to the MHC class I system, 2016 Bioinformatics, 32:511, PMID: 26515819; Jensen K K, Andreatta M, Marcatili P, Buus S, Greenbaum J A, Yan Z, Sette A, Peters B, and Nielsen M, Improved methods for predicting peptide binding affinity to MHC class I molecules, 2018 Immunology, PMID: 29315598). The mutations incorporated into ULBP2.C, ULBP2.R, and ULBP2.AB did not increase predicted immunogenicity while that of ULPB2.AA was increased slightly for a few haplotypes (FIGS. 14 and 15). As a consequence of the specificity of ULBP2.R for NKG2D.AF and its lack of predictable immunogenicity, ULBP2.R was selected for further ELISA analysis to directly compare its binding behavior with that of ULBP2.S3 (the NKG2D.YA-selected, non-natural, orthogonal ligand), ULBP2.R80W (non-natural ligand with enhanced affinity for wild-type NKG2D), and wild-type ULBP2 (ULBP2.wt). Binding of the four rituximab-MicAbody reagents (SEQ ID NOs: 98 and 121, 98 and 129, 131 and 100, and 98 and 106 as heavy chain and light chain for ULBP2.R, ULBP2.S3, ULBP2.R80W, and ULBP2.wt, respectively) was assayed against wild-type NKG2D (NKG2D.wt) and the two inert, non-natural variants NKG2D.YA and NKG2D.AF (FIGS. 16A and 16B). The data demonstrated that NKG2D.YA-selected variant ULBP2.S3 as a MicAbody bound with high affinity to NKG2D.YA but did not engage NKG2D.AF or natural NKG2D. Furthermore, the NKG2D.AF-selected variant ULBP2.R in MicAbody format bound with high affinity to NKG2D.AF but did not engage NKG2D.YA or natural NKG2D. These results demonstrated the tremendous potential of exploring the NKG2D-MIC ligand axis and for developing unique pairs of novel, selective non-natural NKG2D receptors and their respective, cognate non-natural MIC ligand binding partners.

### Example 6: The Targeting and Killing Activity of CAR-T Cells Expressing the Non-Natural NKG2D.AF Ectodomain are Controlled by Orthogonal α1-α2 Domains Fused to Heterologous Targeting Polypeptides

Means to selectively control CAR-T cell therapies are highly sought after to mitigate toxicity and improve efficacy against tumors (Gill and June, op cit). Previous attempts have been made to develop CARs using the ectodomain of CD16 which can then be engaged through the Fc domain of therapeutic monoclonal antibodies, allowing for antibody-based control of CAR-T targeting (Chang et al., op cit). However, CD16-based CAR-T cells can recognize nearly all endogenous antibody molecules in blood and tissues, and the therapeutic antibodies used to control these cells will encounter competition from endogenous CD16 receptors on NK cells, PMN's, monocytes and macrophages. Both of these features contribute problems of off-tumor toxicity and poor pharmacokinetics, respectively.

Natural NKG2D ligands are present on certain healthy tissues and many stressed tissues, creating an extreme risk for toxicity using current NKG2D CAR approaches (VanSeggelen et al. 2015). The Y152A non-natural NKG2D receptor specifically bound to non-natural α1-α2 domain NKG2D ligands constituting an example of a means by which the activity of a non-natural NKG2D CAR could be selectively controlled using bispecific proteins comprised of the invented non-natural α1-α2 domain of NKG2D ligands.

We engineered CAR-T cells with a Receptor comprised of a modified Y152A/Y199F (“AF”) ectodomain of NKG2D which lacks binding to all natural NKG2D ligands or previously described non-natural α1-α2 domains orthogonal and cognate to Y152A modified NKG2D (NKG2D.YA). The invented cognate non-natural α1-α2 domains bound with high affinity to the non-natural NKG2D.AF ectodomain and avoided binding to natural NKG2D ectodomains and to the NKG2D.YA ectodomain. Thus, engineered α1-α2 domains that exhibited strong selectivity for non-natural NKG2D.AF ectodomain over natural NKG2D and non-natural NKG2D.YA represent an ideal system for selective control of non-natural NKG2D CAR receptors, or any receptor or protein fused to non-natural NKG2D ectodomains that can be selectively engaged by the non-natural α1-α2 domains of the instant invention. The instant invention further enables single cells expressing two distinct CARs—one comprised of NKG2D.YA and the other of NKG2D.AF—each signaling with distinctly different intracellular domains. These distinct CARs would possess independent, dual controls of the cell's activities by extracellular exposure to the respective, cognate orthogonal MicAbody or another non-antibody fusion polypeptide.

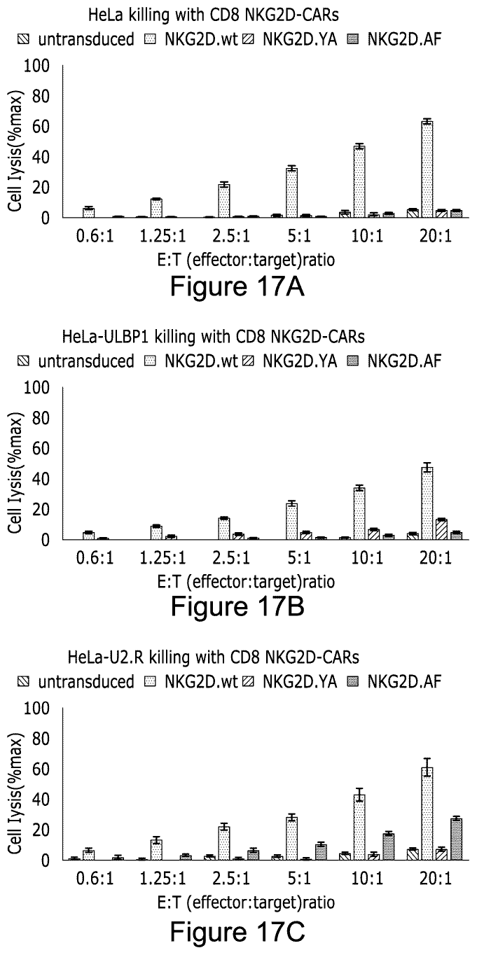

To demonstrate selective control of CAR-T cells constructed with a chimeric receptor deploying the non-natural NKG2D.AF ectodomain, we constructed CARs with either the natural NKG2D.wt (SEQ ID NO: 135), non-natural NKG2D.YA (SEQ ID NO: 137), or the non-natural NKG2D.AF (SEQ ID NO: 139) ectodomains based on previous work using 4-1BB/CD3zeta CAR constructs (Campana patent 8,399,645) fusing the respective NKG2D ectodomains to the CD8 hinge region of CARs (SEQ ID NOs: 151, 153, 155). These constructs (SEQ ID NOs: 152, 154, 156) were cloned into a lentiviral vector and expressed in primary human CD8-positive T cells using lentiviral transduction. HeLa cells have constitutively upregulated levels of MIC ligands on their surface including MICA, MICB, ULBP3, and ULBP2/5/6 (the antibody used to ascertain this cannot distinguish between these three ULBPs; Human ULBP-2/5/6 Antibody, R&D Systems, Minneapolis, MN). HeLa cells were transfected to also over-express either natural ULBP1 or the NKG2D.AF-selected variant ULBP2.R on their surface, and these cells were used as a target for in vitro killing assays. HeLa target cells were pre-loaded with calcein and exposed to NKG2D.wt-CAR, NKG2D.YA-CAR, or NKG2D.AF-CAR CD8 cells at increasing effector to target (E:T) ratios for five hours, after which the amount of calcein released into the supernatant was quantified and normalized to the total calcein released upon detergent treatment (FIGS. 17A-17C). Due to the elevated levels of MIC ligands naturally expressed on the surface of HeLa cells, the CD8 cells expressing natural NKG2D (NKG2D.wt) as the CAR engaged the HeLa cells via this over-expressed natural ligand and effected cytolysis. However, both the NKG2D.YA- and NKG2D.AF-CAR transduced CD8 cells demonstrated very little lysis of natural HeLa cells even at high E:T ratios, a level of activity that is on par with untransduced CD8 T cells. When ULBP1 is overexpressed on the surface of HeLa cells, only the NKG2D.wt-CAR CD8 T cells significantly lysed them. There is some additional killing at high E:T ratio with NKG2D.YA-CAR cells, but this is non-existent with NKG2D.AF-CAR cells showing that the double mutation Y152A/Y199F renders NKG2D even more inert than the single Y152A mutation. In HeLa cells over-expressing the NKG2D.AF-selective non-natural ULBP2.R, NKG2D.wt-CAR cells direct lysis (due to recognition of endogenous MIC ligands) while NKG2D.AF-CAR cells directed significant levels of lysis consistent with engagement of the receptor and its selective ligand.

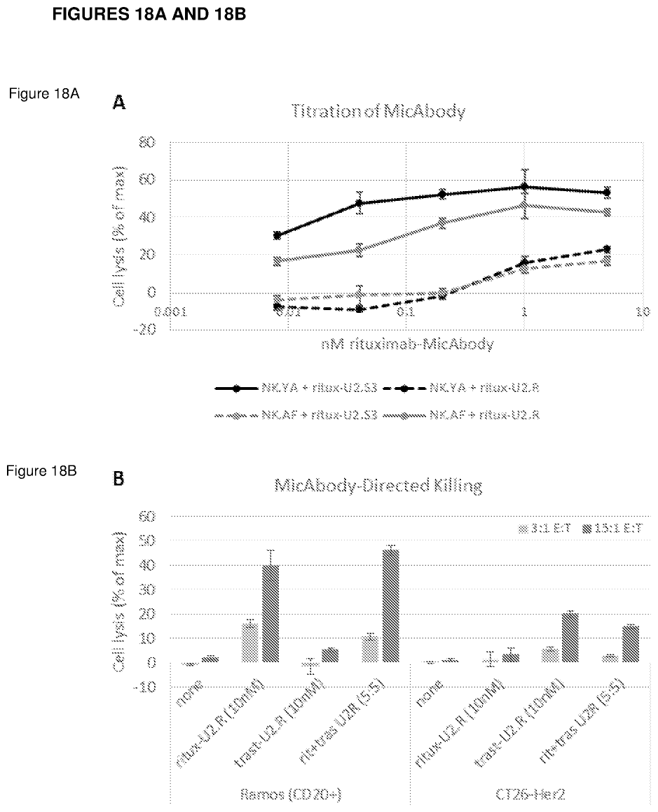

In order to demonstrate that lysis of either NKG2D.YA- or NKG2D.AF-CAR cells could only be directed by the appropriate, cognate targeting MicAbody, Ramos cells were used as a target for cytolysis in combination with rituximab-based MicAbodies linked to either non-natural ULBP2.S3 or ULBP2.R orthogonal ligands. As demonstrated in FIG. 18A, the rituximab-ULBP2.S3 MicAbody could direct the cell killing activity of NKG2D.YA-CAR CD8 cells but not NKG2D.AF-CAR cells, while the rituximab-ULBP2.R MicAbody could direct the activity of NKG2D.AF-CAR but not NKG2D.YA-CAR cells. This further demonstrates the selectivity of the two non-natural ULBP2 variants for their cognate non-natural NKG2D variants for which they were engineered as preferred partners. In order to demonstrate the specificity of the antibody portion of the MicAbody, in vitro killing assays were performed with NKG2D.AF-CAR CD8 cells that were pre-armed by incubation with either rituximab-ULBP2.R, trastuzumab-ULPB2.R (SEQ ID NOs: 95 and 133, heavy and light chain, respectively), or an equimolar combination of the two at a saturating total concentration of MicAbody. After unbound MicAbody was removed by washing, the CD8 cells were applied to either Ramos cells (expressing CD20, the target of rituximab) or to CT26-Her2 (a mouse cell line transfected to express human Her2) that had been pre-loaded with calcein. After a two hour incubation at two different E:T ratios, the amount of calcein released was quantified. As shown in FIG. 18B, when cells were pre-armed with rituximab-MicAbody, only Ramos cells were lysed while trastuzumab-MicAbody directed cytolytic activity only against CT26-Her2 cells. However, when NKG2D.AF-CAR CD8 cells were simultaneously pre-armed with both rituximab- and trastuzumab-ULBP2.R MicAbodies, both target cells lines were lysed demonstrating that these CAR cells—by virtue of the selective, privileged partnering that has been engineered between receptor and ligand—were readily multiplexed and thereby directed to engage different tumor targets simultaneously.

What is claimed is:

1. A non-natural, modified α1-α2 domain of a natural NKG2D ligand molecule comprising at least 80% identity to the natural α1-α2 domain, wherein the modified α1-α2 domain has more than 5 amino acids of the natural α1-α2 domain replaced and wherein the modification increases its binding affinity to a non-natural NKG2D receptor ectodomain, wherein the ectodomain of the non-natural NKG2D receptor has an affinity for at least one natural NKG2D ligand that is less than the affinity of the natural NKG2D receptor ectodomain for the at least natural NKG2D ligand.

2. The non-natural, modified α1-α2 domain of claim 1, wherein the at least one natural NKG2D ligand is any one of SEQ ID NO.s: 1-9.

3. The non-natural, modified α1-α2 domain of claim 1, wherein the amino acids that are replaced from the natural α1-α2 domain are at three or more of positions 8, 80, 154, 155, 156, 157, 158, and 159 of SEQ ID NO.: 4.

4. The non-natural, modified α1-α2 domain of claim 3, which comprises an amino acid sequence selected from the group consisting of SEQ ID NOs: 111, 113, 115 and 117.

5. The non-natural, modified α1-α2 domain of claim 1, wherein said non-natural NKG2D receptor ectodomain comprises the amino acid sequence of SEQ ID NO: 17 but in which the tyrosines at position 75 and position 122 of SEQ ID NO: 17 have both been replaced with another amino acid.

6. The non-natural, modified α1-α2 domain of claim 5, wherein the amino acid replacing the tyrosine at position 75 is alanine, and wherein the amino acid replacing the tyrosine at position 122 is phenylalanine.

7. The non-natural, modified α1-α2 domain of claim 1, further comprising an attached atom or an attached heterologous molecule thereby creating a bispecific molecule.

8. The non-natural, modified α1-α2 domain of claim 7, wherein the heterologous molecule is a peptide or a polypeptide.

9. The non-natural, modified α1-α2 domain of claim 8, wherein the polypeptide is an antibody, antibody fragment, cytokine, lymphokine, or hormone.

10. The non-natural, modified α1-α2 domain of claim 7, wherein the heterologous molecule is an oligosaccharide, dendrimer, knottin, hormone, nucleic acid, or lipid.

11. A mammalian cell to which the non-natural NKG2D receptor ectodomain of claim 1 is attached.

12. The mammalian cell of claim 11, wherein the mammalian cell is a lymphocyte or a macrophage.

13. The mammalian cell of claim 12, wherein the mammalian cell is a human lymphocyte or a human macrophage.

14. The non-natural, modified α1-α2 domain of claim 7, wherein when said non-natural, modified α1-α2 domain is contacted with a mammalian cell having a non-natural NKG2D receptor ectodomain attached thereto, said atom or heterologous molecule is delivered to said mammalian cell.

15. The non-natural, modified α1-α2 domain of claim 14, which exhibits an enhanced activation of a cell expressing the non-natural NKG2D ectodomain resulting in the cell exhibiting greater target cell killing potency than does a natural NKG2D ligand or a bispecific molecule with a natural or native α1-α2 domain delivered to the cell.

16. The non-natural, modified α1-α2 domain of claim 14, which exhibits greater activation of a cell expressing the non-natural NKG2D ectodomain than activation of a cell expressing a natural NKG2D ectodomain resulting in the cell expressing the non-natural NKG2D ectodomain exhibiting greater target cell killing potency than does a cell expressing the natural NKG2D ectodomain.
